(19) United States
(12) Patent Application Publication
(10) Pub. No.: US 2026/0224834 A1
(43) Pub. Date: Aug. 6, 2026

(54) SYSTEMS AND METHODS FOR IDENTIFYING BREATHING COMFORT

(71) Applicant: ResMed Digital Health Inc., San Diego, CA (US)

(72) Inventors: Gregory Robert PEAKE, San Diego, CA (US); Nathan Zersee LIU, Sydney (AU); Genevieve Claire ATKINSON, Sydney (AU); Chinh Duc NGUYEN, Sydney (AU); Jaiden James CHOY, Sydney (AU); Brian Baillie MACLEOD, Nova Scotia (CA); Harley Brett KOLTAI, Sydney (AU); Anna RICE, Singapore (SG); Kristy Tzu Ying TAN, Sydney (AU)

(21) Appl. No.: 19/150,150

(22) PCT Filed: Jan. 23, 2024
(86) PCT No.: PCT/US2024/012648

§ 371 Date: Jul. 22, 2025

Related U.S. Application Data
(60) Provisional application No. 63/481,229, filed on Jan. 24, 2023.

(51) Int. Cl. A61M 16/00

(52) U.S. Cl. CPC A61M 16/024; A61M 2205/3334; A61M 2205/3344; A61M 2205/3576; A61M 2205/502; A61M 2230/005

(57) ABSTRACT

A method for classifying a user of a respiratory pressure therapy (RPT) device is disclosed, the method comprising: identifying that the user has commenced a therapy session with the RPT device; receiving first input data associated with the user and the therapy session; training a machine learning model based on a plurality of pre-classified users and the first input data to determine an association between a user and a plurality of classifications; classifying the user into a classification of the plurality of classifications; and in response to classifying the user into the classification, causing a user device and/or the RPT device to execute an action. The first input data may include selections from the user in response to a survey prompt.

### RELATED APPLICATIONS

This present application claims the benefit of priority under 35 U.S.C. § 119(e) to U.S. Provisional Application No. 63/481,229, entitled “SYSTEMS AND METHODS FOR IDENTIFYING BREATHING COMFORT”, filed Jan. 24, 2023, the disclosure of which is hereby incorporated by reference in its entirety for all purposes.

### TECHNICAL FIELD

The present technology relates to one or more of the screening, diagnosis, monitoring, treatment, prevention, and amelioration of respiratory-related disorders. The present technology also relates to medical devices or apparatus, and their use.

### BACKGROUND

Many individuals suffer from sleep-related and/or respiratory disorders. Examples of sleep-related and/or respiratory disorders include Periodic Limb Movement Disorder (PLMD), Restless Leg Syndrome (RLS), Sleep-Disordered Breathing (SDB), Obstructive Sleep Apnea (OSA), apneas, Cheyne-Stokes Respiration (CSR), respiratory insufficiency, Obesity Hyperventilation Syndrome (OHS), Chronic Obstructive Pulmonary Disease (COPD), Neuromuscular Disease (NMD), and chest wall disorders.

These and other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping.

In order to mitigate some of these sleep-related and/or respiratory disorders, a user can be prescribed usage of a respiratory device or system. For example, a continuous positive airway pressure (CPAP) machine can be used to increase air pressure in the throat of the respiratory device user and to prevent the airway from closing and/or narrowing during sleep. While these respiratory devices or systems can improve the sleep quality of the user, the comfort of the user is sometimes compromised. For example, while attempting to sleep, a user may have some discomfort, including a sensation of a lack of air when first wearing a mask associated with the respiratory device, or other discomfort due to air being forced into their airways.

The discomfort experienced by a user of a respiratory therapy system is often more pronounced when the user is first adopting a sleep therapy using the system. If the initial user experience is unsatisfactory or uncomfortable, the user is more likely to abandon the therapy and sacrifice sleep quality in pursuit of better sleep comfort. Deferment of treatment can lead to deterioration of quality of life due to the user not achieving high quality sleep. Therefore, there exists a need to help users achieve comfort, including early in their treatment, and to identify users who may be experiencing discomfort so that interventions may be offered to improve the comfort of their treatment.

### SUMMARY

In one aspect of the disclosure, a computer-implemented method for classifying a user of a respiratory pressure therapy (RPT) device is disclosed, the method comprising: identifying that the user has commenced a therapy session with the RPT device; receiving first input data associated with the user and the therapy session; training a machine learning model based on a plurality of pre-classified users and the first input data to determine an association between a user and a plurality of classifications; classifying the user into a classification of the plurality of classifications; and in response to classifying the user into the classification, causing a user device and/or the RPT device to execute an action.

In another aspect of the disclosure, a system for classifying a user of a respiratory pressure therapy (RPT) device is disclosed, the system comprising: a memory storing instructions and a machine learning model trained based on pre-classified users so as to learn associations between the pre-classified users and a plurality of classifications, such that the machine learning model is configured to perform the classifying based on the learned associations; and a processor operatively connected to the memory and configured to execute the instructions to perform operations including: identifying that the user has commenced a therapy session on the RPT device; receiving first input data associated with the user and the therapy session; classifying the user into a classification of the plurality of classifications by inputting the first input data into the machine learning model; and in response to classifying the user into the classification, causing the user device and/or the RPT device to execute an action.

In yet another aspect of the disclosure, a non-transient computer-readable storage medium having instructions is disclosed. The instructions are executable by one or more processors to perform a method for training a machine-learning model for classifying a user of a respiratory pressure therapy (RPT) device, the method comprising: receiving input data associated with users and associated therapy sessions. Each user has one or more classifications. The method further comprises processing the input data to extract one or more features from the input data; training the machine learning model to classify one or more other users into the one or more classifications, based on the one or more feature extracted from the input data; and providing communication between the machine learning model and one or both of a user device and the RPT device, wherein the communication comprises at least causing a user device and/or the RPT device to execute an action.

The methods, systems, devices and apparatus described may be implemented so as to improve the functionality of a processor, such as a processor of a specific purpose computer, respiratory monitor and/or a respiratory therapy apparatus. Moreover, the described methods, systems, devices and apparatus can provide improvements in the technological field of automated management, monitoring and/or treatment of respiratory conditions, including, for example, sleep disordered breathing.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### BRIEF DESCRIPTION OF THE DRAWINGS

FIG. 1 is a functional block diagram of a system, according to some implementations of the present disclosure.

FIG. 2 is a perspective view of at least a portion of the system of FIG. 1, a user, and a bed partner, according to some implementations of the present disclosure.

FIG. 3 is a plot of a ramp feature and a respiratory flow rate, according to some implementations of the present disclosure.

FIG. 4A is a block diagram illustrating a respiratory pressure therapy system according to one implementation of the present disclosure.

FIG. 4B is a block diagram illustrating a respiratory pressure therapy system according to another implementation of the present disclosure.

FIG. 5A is an exemplary graphic user interface (GUI) depicting a first sleep survey window, according to some implementations of the present disclosure.

FIG. 5B is an exemplary GUI depicting a second sleep survey window, according to some implementations of the present disclosure.

FIG. 6 is an exemplary GUI depicting instructions to a user to turn ramp on or off, according to some implementations of the present disclosure.

FIG. 7 is an exemplary GUI depicting instructions to a user to turn EPR on or off, according to some implementations of the present disclosure.

FIG. 8 is an exemplary GUI depicting a sleep survey follow-up, according to some implementations of the present disclosure.

FIG. 9 shows a model typical breath waveform of a person while sleeping.

FIG. 10 depicts a flowchart of an exemplary process for training a machine-learning model to learn associations between user data and classifications of the user, according to some embodiments.

FIG. 11 depicts a flowchart of an exemplary process for classifying users based on a trained machine-learning model, according to some embodiments.

FIG. 12 depicts a flowchart of an alternative exemplary process for classifying users based on a trained machine-learning model, according to some embodiments.

### DETAILED DESCRIPTION OF EMBODIMENTS

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

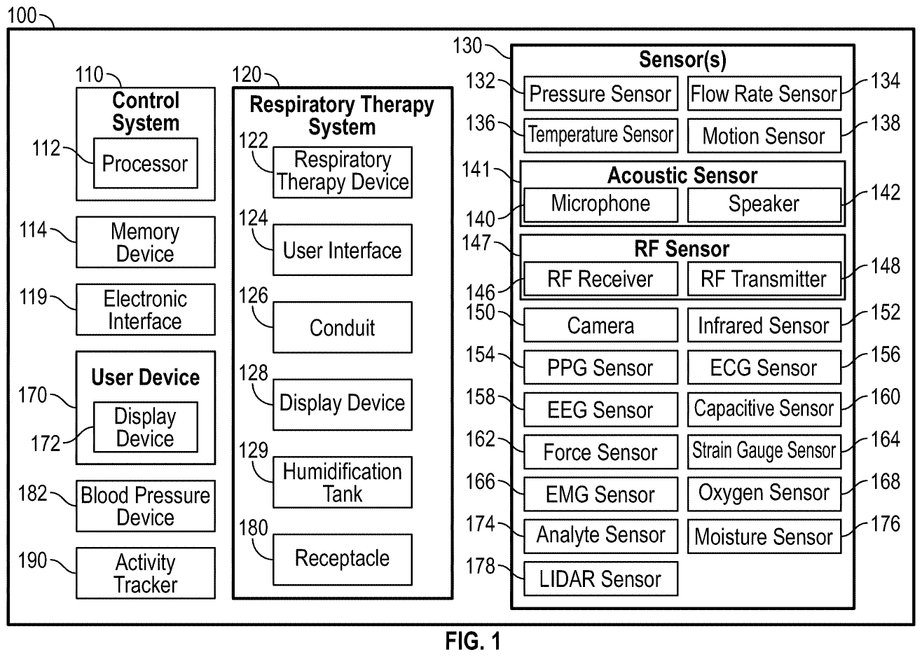

Referring to FIG. 1, a system 100, according to some implementations of the present disclosure, is illustrated. The system 100 includes a control system 110, a memory device 114, an electronic interface 119, a respiratory therapy system 120, one or more sensors 130, and one or more user devices 170. In some implementations, the system 100 further includes a blood pressure device 182, an activity tracker 190, or both.

The control system 110 includes one or more processors 112 (hereinafter, processor 112). The control system 110 is generally used to control (e.g., actuate) the various components of the system 100 and/or analyze data obtained and/or generated by the components of the system 100. The processor 112 can be a general or special purpose processor or microprocessor. While one processor 112 is shown in FIG. 1, the control system 110 can include any suitable number of processors (e.g., one processor, two processors, five processors, ten processors, etc.) that can be in a single housing, or located remotely from each other. The control system 110 can be coupled to and/or positioned within, for example, a housing of the user device 170, and/or within a housing of one or more of the sensors 130 or the respiratory therapy system 120. The control system 110 can be centralized (within one such housing) or decentralized (within two or more of such housings, which are physically distinct). In such implementations including two or more housings containing the control system 110, such housings can be located proximately and/or remotely from each other.

The memory device 114 stores machine-readable instructions that are executable by the processor 112 of the control system 110. The memory device 114 can be any suitable computer readable storage device or media, such as, for example, a random or serial access memory device, a hard drive, a solid state drive, a flash memory device, etc. While one memory device 114 is shown in FIG. 1, the system 100 can include any suitable number of memory devices 114 (e.g., one memory device, two memory devices, five memory devices, ten memory devices, etc.). The memory device 114 can be coupled to and/or positioned within a housing of a RPT device 122, within a housing of the user device 170, within a housing of one or more of the sensors 130, or any combination thereof. Like the control system 110, the memory device 114 can be centralized (within one such housing) or decentralized (within two or more of such housings, which are physically distinct).

In some implementations, the memory device 114 (FIG. 1) stores a user profile associated with a user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a geographic location of the user, a relationship status, a family history of insomnia or sleep apnea, an employment status of the user, an educational status of the user, a socioeconomic status of the user, or any combination thereof. The medical information can include, for example, information indicative of one or more medical conditions associated with the user, medication usage by the user, or both. The medical information data can further include a multiple sleep latency test (MSLT) result or score and/or a Pittsburgh Sleep Quality Index (PSQI) score or value. The self-reported user feedback can include information indicative of a self-reported subjective sleep score (e.g., poor, average, excellent), a self-reported subjective stress level of the user, a self-reported subjective fatigue level of the user, a self-reported subjective health status of the user, a recent life event experienced by the user, or any combination thereof.

The electronic interface 119 is configured to receive data (e.g., physiological data and/or acoustic data) from the one or more sensors 130 such that the data can be stored in the memory device 114 and/or analyzed by the processor 112 of the control system 110. The electronic interface 119 can communicate with the one or more sensors 130 using a wired connection or a wireless connection (e.g., using an RF communication protocol, a WiFi communication protocol, a Bluetooth communication protocol, over a cellular network, etc.).

The electronic interface 119 can include an antenna, a receiver (e.g., an RF receiver), a transmitter (e.g., an RF transmitter), a transceiver, or any combination thereof. The electronic interface 119 can also include one more processors and/or one more memory devices that are the same as, or similar to, the processor 112 and the memory device 114 described herein. In some implementations, the electronic interface 119 is coupled to or integrated in the user device 170. In other implementations, the electronic interface 119 is coupled to or integrated (e.g., in a housing) with the control system 110 and/or the memory device 114.

As noted above, in some implementations, the system 100 includes a respiratory therapy system 120. The respiratory therapy system 120 may include a respiratory pressure therapy (RPT) device 122 (referred to herein as RPT device 122), a user interface 124, a conduit 126 (also referred to as a tube or an air circuit), a display device 128, a humidification tank 129, a receptacle 180, or any combination thereof. In some implementations, the control system 110, the memory device 114, the display device 128, one or more of the sensors 130, and the humidification tank 129 are part of the RPT device 122. Respiratory pressure therapy refers to the application of a supply of air to an entrance of the user's airways at a controlled target pressure that is nominally positive with respect to atmosphere throughout the user's respiratory cycle (e.g., in contrast to negative pressure therapies such as the tank ventilator or cuirass). The respiratory therapy system 120 is generally used to treat individuals suffering from one or more sleep-related respiratory disorders (e.g., obstructive sleep apnea, central sleep apnea, or mixed sleep apnea).

The RPT device 122 has a blower motor (not shown) that is generally used to generate pressurized air that is delivered to the user (e.g., using one or more motors that drive one or more compressors). In some implementations, the RPT device 122 generates continuous constant air pressure that is delivered to the user. In other implementations, the RPT device 122 generates two or more predetermined pressures (e.g., a first predetermined air pressure and a second predetermined air pressure). In still other implementations, the RPT device 122 is configured to generate a variety of different air pressures within a predetermined range. For example, the RPT device 122 can deliver at least about 4 cm H2O, at least about 10 cm H2O, at least about 20 cm H2O, between about 6 cm H2O and about 10 cm H2O, between about 7 cm H2O and about 12 cm H2O, etc. The RPT device 122 can also deliver pressurized air at a predetermined flow rate between, for example, about −20 liters/minute and about 150 liters/minute, while maintaining a positive pressure (relative to the ambient pressure).

The user interface 124 engages a portion of the user's face and delivers pressurized air from the RPT device 122 to the user's airway to aid in preventing the airway from narrowing and/or collapsing during sleep. Generally, the user interface 124 engages the user's face such that the pressurized air is delivered to the user's airway via the user's mouth, the user's nose, or both the user's mouth and nose. Together, the RPT device 122, the user interface 124, and the conduit 126 form an air pathway fluidly coupled with an airway of the user. The pressurized air also increases the user's oxygen intake during sleep. Depending upon the therapy to be applied, the user interface 124 may form a seal, for example, with a region or portion of the user's face, to facilitate the delivery of air at a pressure at sufficient variance with ambient pressure to effect therapy, for example, at a positive pressure of about 10 cm H2O relative to ambient pressure. For other forms of therapy, such as the delivery of oxygen, the user interface may not include a seal sufficient to facilitate delivery to the airways of a supply of gas at a positive pressure of about 10 cm H2O.

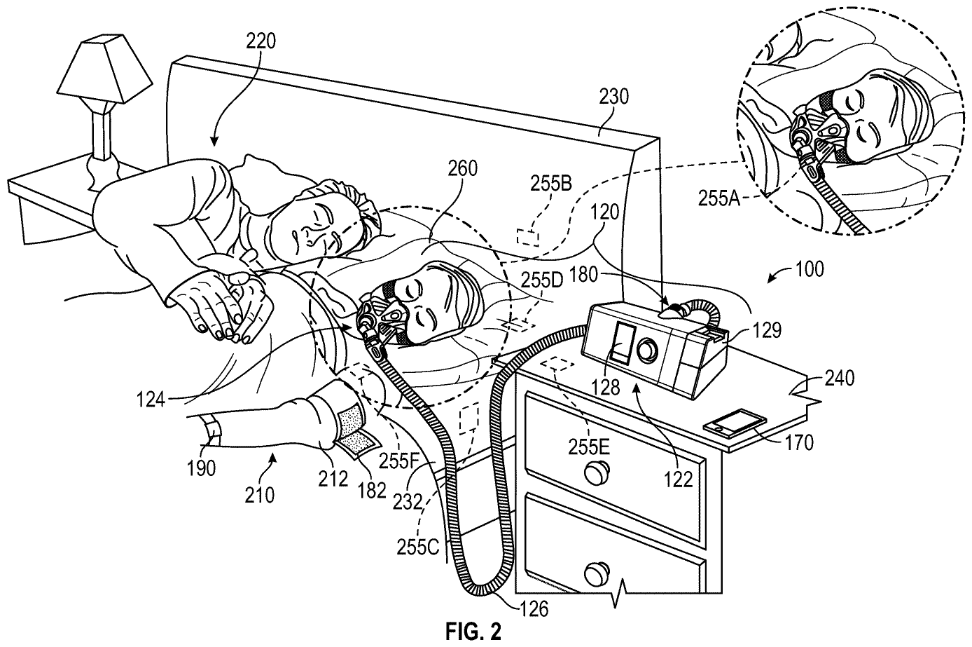

As shown in FIG. 2, in some implementations, the user interface 124 is a facial mask (e.g., a full facial mask) that covers the nose and mouth of a patient or user 210. Throughout this Specification, the terms “patient” and “user” may be understood to be used interchangeably as a user of the respiratory pressure therapy device. Alternatively, the user interface 124 can be a nasal mask that provides air to the nose of the user 210 or a nasal pillow mask that delivers air directly to the nostrils of the user 210. The user interface 124 can include a plurality of straps forming, for example, a headgear for aiding in positioning and/or stabilizing the interface on a portion of the user 210 (e.g., the face) and a conformal cushion (e.g., silicone, plastic, foam, etc.) that aids in providing an air-tight seal between the user interface 124 and the user 210. The user interface 124 can also include one or more vents for permitting the escape of carbon dioxide and other gases exhaled by the user 210. In other implementations, the user interface 124 includes a mouthpiece (e.g., a night guard mouthpiece molded to conform to the teeth of the user 210, a mandibular repositioning device, etc.).

The conduit 126 (also referred to as an air circuit or tube) allows the flow of air between two components of a respiratory therapy system 120, such as the RPT device 122 and the user interface 124. In some implementations, there can be separate limbs of the conduit 126 for inhalation and exhalation. In other implementations, a single limb conduit is used for both inhalation and exhalation.

One or more of the RPT device 122, the user interface 124, the conduit 126, the display device 128, and the humidification tank 129 can contain one or more sensors (e.g., a pressure sensor, a flow rate sensor, or more generally any of the other sensors 130 described herein). These one or more sensors can be used, for example, to measure the air pressure, flow rate, and/or other parameters of pressurized air supplied by the RPT device 122.

The display device 128 is generally used to display image(s) including still images, video images, or both, and/or information regarding the RPT device 122. For example, the display device 128 can provide information regarding the status of the RPT device 122 (e.g., whether the RPT device 122 is on/off, the pressure, temperature, and/or other parameters of the air being delivered by the respiratory device 122, etc.) and/or other information (e.g., a sleep score and/or a therapy score, also referred to as a my Air™ score, such as described in US 2017/0311879 A1, which is hereby incorporated by reference herein in its entirety, the current date/time, personal information for the user 210, questions seeking feedback from the user 210 and/or advice to the user 210, etc.). In some implementations, the display device 128 acts as a human-machine interface (HMI) that includes a graphic user interface (GUI) configured to display the image(s) as an input interface. The display device 128 can be an LED display, an OLED display, an LCD display, or the like. The input interface can be, for example, a touchscreen or touch-sensitive substrate, a mouse, a keyboard, or any sensor system configured to sense inputs made by a human user interacting with the RPT device 122.

The humidification tank 129 is coupled to, or integrated in, the RPT device 122 and includes a reservoir of water that can be used to humidify the pressurized air delivered from the RPT device 122. The RPT device 122 can include one or more vents (not shown) and a heater to heat the water in the humidification tank 129 in order to humidify the pressurized air provided to the user 210. Additionally, in some implementations, the conduit 126 can also include a heating element (e.g., coupled to and/or imbedded in the conduit 126) that heats the pressurized air delivered to the user 210. The humidification tank 129 can be fluidly coupled to a water vapor inlet of the air pathway and deliver water vapor into the air pathway via the water vapor inlet, or can be formed in-line with the air pathway as part of the air pathway itself. In some implementations, the humidification tank 129 may not include the reservoir of water and thus waterless.

In some implementations, the system 100 can be used to deliver at least a portion of a substance from the receptacle 180 to the air pathway of the user based at least in part on the physiological data, the sleep-related parameters, other data or information, or any combination thereof. Generally, modifying the delivery of the portion of the substance into the air pathway can include (i) initiating the delivery of the substance into the air pathway, (ii) ending the delivery of the portion of the substance into the air pathway, (iii) modifying an amount of the substance delivered into the air pathway, (iv) modifying a temporal characteristic of the delivery of the portion of the substance into the air pathway, (v) modifying a quantitative characteristic of the delivery of the portion of the substance into the air pathway, (vi) modifying any parameter associated with the delivery of the substance into the air pathway, or (vii) a combination of (i)-(vi).

Modifying the temporal characteristic of the delivery of the portion of the substance into the air pathway can include changing the rate at which the substance is delivered, starting and/or finishing at different times, continuing for different time periods, changing the time distribution or characteristics of the delivery, changing the amount distribution independently of the time distribution, etc. The independent time and amount variation ensures that, apart from varying the frequency of the release of the substance, one can vary the amount of substance released each time. In this manner, a number of different combination of release frequencies and release amounts (e.g., higher frequency but lower release amount, higher frequency and higher amount, lower frequency and higher amount, lower frequency and lower amount, etc.) can be achieved. Other modifications to the delivery of the portion of the substance into the air pathway can also be utilized.

The respiratory therapy system 120 can be used, for example, as a ventilator or as a positive airway pressure (PAP) system, such as a continuous positive airway pressure (CPAP) system, an automatic positive airway pressure system (APAP), a bi-level or variable positive airway pressure system (BPAP or VPAP), or any combination thereof. The CPAP system delivers a predetermined amount of pressurized air (e.g., determined by a sleep physician) to the user 210. The APAP system automatically varies the pressurized air delivered to the user 210 based on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

Still referring to FIG. 1, the one or more sensors 130 of the system 100 include a pressure sensor 132, a flow rate sensor 134, temperature sensor 136, a motion sensor 138, a microphone 140, a speaker 142, a radio-frequency (RF) receiver 146, a RF transmitter 148, a camera 150, an infrared sensor 152, a photoplethysmogram (PPG) sensor 154, an electrocardiogram (ECG) sensor 156, an electroencephalography (EEG) sensor 158, a capacitive sensor 160, a force sensor 162, a strain gauge sensor 164, an electromyography (EMG) sensor 166, an oxygen sensor 168, an analyte sensor 174, a moisture sensor 176, a LiDAR sensor 178, or any combination thereof. Generally, each of the one or more sensors 130 is configured to output sensor data that is received and stored in the memory device 114 or one or more other memory devices.

While the one or more sensors 130 are shown and described as including each of the pressure sensor 132, the flow rate sensor 134, the temperature sensor 136, the motion sensor 138, the microphone 140, the speaker 142, the RF receiver 146, the RF transmitter 148, the camera 150, the infrared sensor 152, the photoplethysmogram (PPG) sensor 154, the electrocardiogram (ECG) sensor 156, the electroencephalography (EEG) sensor 158, the capacitive sensor 160, the force sensor 162, the strain gauge sensor 164, the electromyography (EMG) sensor 166, the oxygen sensor 168, the analyte sensor 174, the moisture sensor 176, and the LiDAR sensor 178, more generally, the one or more sensors 130 can include any combination and any number of each of the sensors described and/or shown herein.

As described herein, the system 100 generally can be used to generate physiological data associated with a user (e.g., a user of the respiratory therapy system 120 shown in FIG. 2) during a sleep session. The physiological data can be analyzed to generate one or more sleep-related parameters, which can include any parameter, measurement, etc. related to the user during the sleep session. The one or more sleep-related parameters that can be determined for the user 210 during the sleep session include, for example, an Apnea-Hypopnea Index (AHI) score, a sleep score, a flow signal, a pressure signal, a respiration signal, a respiration rate, an inspiration amplitude, an expiration amplitude, an inspiration-expiration ratio, a number of events per hour, a pattern of events, a stage, pressure settings of the RPT device 122, a heart rate, a heart rate variability, movement of the user 210, temperature, EEG activity, EMG activity, arousal, snoring, choking, coughing, whistling, wheezing, or any combination thereof.

The one or more sensors 130 can be used to generate, for example, physiological data, acoustic data, or both. Physiological data generated by one or more of the sensors 130 can be used by the control system 110 to determine a sleep-wake signal associated with the user 210 (FIG. 2) during the sleep session and one or more sleep-related parameters. The sleep-wake signal can be indicative of one or more sleep states, including wakefulness, relaxed wakefulness, micro-awakenings, or distinct sleep stages such as, for example, a rapid eye movement (REM) stage, a first non-REM stage (often referred to as “N1”), a second non-REM stage (often referred to as “N2”), a third non-REM stage (often referred to as “N3”), or any combination thereof.

In some implementations, the sleep-wake signal described herein can be timestamped to indicate a time that the user enters the bed, a time that the user exits the bed, a time that the user attempts to fall asleep, etc. The sleep-wake signal can be measured by the one or more sensors 130 during the sleep session at a predetermined sampling rate, such as, for example, one sample per second, one sample per 30 seconds, one sample per minute, etc. In some implementations, the sleep-wake signal can also be indicative of a respiration signal, a respiration rate, an inspiration amplitude, an expiration amplitude, an inspiration-expiration ratio, a number of events per hour, a pattern of events, pressure settings of the RPT device 122, or any combination thereof during the sleep session. The event(s) can include snoring, apneas, central apneas, obstructive apneas, mixed apneas, hypopneas, a mask leak (e.g., from the user interface 124), a restless leg, a sleeping disorder, choking, an increased heart rate, labored breathing, an asthma attack, an epileptic episode, a seizure, or any combination thereof. The one or more sleep-related parameters that can be determined for the user during the sleep session based on the sleep-wake signal include, for example, a total time in bed, a total sleep time, a sleep onset latency, a wake-after-sleep-onset parameter, a sleep efficiency, a fragmentation index, or any combination thereof. As described in further detail herein, the physiological data and/or the sleep-related parameters can be analyzed to determine one or more sleep-related scores.

Generally, the sleep session includes any point in time after the user 210 has laid or sat down in the bed 230 (or another area or object on which they intend to sleep), and/or has turned on the respiratory device 122 and/or donned the user interface 124. The sleep session can thus include time periods (i) when the user 210 is using the CPAP system but before the user 210 attempts to fall asleep (for example when the user 210 lays in the bed 230 reading a book); (ii) when the user 210 begins trying to fall asleep but is still awake; (iii) when the user 210 is in a light sleep (also referred to as stage 1 and stage 2 of non-rapid eye movement (NREM) sleep); (iv) when the user 210 is in a deep sleep (also referred to as slow-wave sleep, SWS, or stage 3 of NREM sleep); (v) when the user 210 is in rapid eye movement (REM) sleep; (vi) when the user 210 is periodically awake between light sleep, deep sleep, or REM sleep; or (vii) when the user 210 wakes up and does not fall back asleep.

The sleep session is generally defined as ending once the user 210 removes the user interface 124, turns off the respiratory device 122, and/or gets out of bed 230. In some implementations, the sleep session can include additional periods of time, or can be limited to only some of the above-disclosed time periods. For example, the sleep session can be defined to encompass a period of time beginning when the respiratory device 122 begins supplying the pressurized air to the airway or the user 210, ending when the respiratory device 122 stops supplying the pressurized air to the airway of the user 210, and including some or all of the time points in between, when the user 210 is asleep or awake.

The pressure sensor 132 outputs pressure data that can be stored in the memory device 114 and/or analyzed by the processor 112 of the control system 110. In some implementations, the pressure sensor 132 is an air pressure sensor (e.g., barometric pressure sensor) that generates sensor data indicative of the respiration (e.g., inhaling and/or exhaling) of the user of the respiratory therapy system 120 and/or ambient pressure. In such implementations, the pressure sensor 132 can be coupled to or integrated in the RPT device 122. The pressure sensor 132 can be, for example, a capacitive sensor, an electromagnetic sensor, a piezoelectric sensor, a strain-gauge sensor, an optical sensor, a potentiometric sensor, or any combination thereof.

The flow rate sensor 134 outputs flow rate data that can be stored in the memory device 114 and/or analyzed by the processor 112 of the control system 110. In some implementations, the flow rate sensor 134 is used to determine an air flow rate from the RPT device 122, an air flow rate through the conduit 126, an air flow rate through the user interface 124, or any combination thereof. In such implementations, the flow rate sensor 134 can be coupled to or integrated in the RPT device 122, the user interface 124, or the conduit 126. The flow rate sensor 134 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof. In some implementations, the flow rate sensor 134 is configured to measure a vent flow (e.g., intentional “leak”), an unintentional leak (e.g., mouth leak and/or mask leak), a patient flow (e.g., air into and/or out of lungs), or any combination thereof. In some implementations, the flow rate data can be analyzed to determine cardiogenic oscillations of the user. In one example, the pressure sensor 132 can be used to determine a blood pressure of a user.

The temperature sensor 136 outputs temperature data that can be stored in the memory device 114 and/or analyzed by the processor 112 of the control system 110. In some implementations, the temperature sensor 136 generates temperatures data indicative of a core body temperature of the user 210 (FIG. 2), a skin temperature of the user 210, a temperature of the air flowing from the RPT device 122 and/or through the conduit 126, a temperature in the user interface 124, an ambient temperature, or any combination thereof. The temperature sensor 136 can be, for example, a thermocouple sensor, a thermistor sensor, a silicon band gap temperature sensor or semiconductor-based sensor, a resistance temperature detector, or any combination thereof.

The motion sensor 138 outputs motion data that can be stored in the memory device 114 and/or analyzed by the processor 112 of the control system 110. The motion sensor 138 can be used to detect movement of the user 210 during the sleep session, and/or detect movement of any of the components of the respiratory therapy system 120, such as the RPT device 122, the user interface 124, or the conduit 126. The motion sensor 138 can include one or more inertial sensors, such as accelerometers, gyroscopes, and magnetometers. In some implementations, the motion sensor 138 alternatively or additionally generates one or more signals representing bodily movement of the user, from which may be obtained a signal representing a sleep state of the user; for example, via a respiratory movement of the user. In some implementations, the motion data from the motion sensor 138 can be used in conjunction with additional data from another sensor 130 to determine the sleep state of the user.

The microphone 140 outputs sound and/or audio data that can be stored in the memory device 114 and/or analyzed by the processor 112 of the control system 110. The audio data generated by the microphone 140 is reproducible as one or more sound(s) during a sleep session (e.g., sounds from the user 210). The audio data from the microphone 140 can also be used to identify (e.g., using the control system 110) one or more sleep-related parameters or events experienced by the user 210 during the sleep session, as described in further detail herein. The microphone 140 can be coupled to or integrated in the RPT device 122, the user interface 124, the conduit 126, or the user device 170. In some implementations, the system 100 includes a plurality of microphones (e.g., two or more microphones and/or an array of microphones with beamforming) such that sound data generated by each of the plurality of microphones can be used to discriminate the sound data generated by another of the plurality of microphones.

The speaker 142 outputs sound waves that are audible to a user of the system 100 (e.g., the user 210 of FIG. 2). The speaker 142 can be used, for example, as an alarm clock or to play an alert or message to the user 210 (e.g., in response to an event). In some implementations, the speaker 142 can be used to communicate the audio data generated by the microphone 140 to the user 210. The speaker 142 can be coupled to or integrated in the RPT device 122, the user interface 124, the conduit 126, or the user device 170.

The microphone 140 and the speaker 142 can be used as separate devices. In some implementations, the microphone 140 and the speaker 142 can be combined into an acoustic sensor 141, (e.g., a sonar sensor). In such implementations, the speaker 142 generates or emits sound waves at a predetermined interval and the microphone 140 detects the reflections of the emitted sound waves from the speaker 142. The sound waves generated or emitted by the speaker 142 have a frequency that is not audible to the human ear (e.g., below 20 Hz or above around 18 kHz) so as not to disturb the sleep of the user 210 or the bed partner 220 (FIG. 2). Based at least in part on the data from the microphone 140 and/or the speaker 142, the control system 110 can determine a location of the user 210 (FIG. 2) and/or one or more of the sleep-related parameters described in herein such as, for example, a respiration signal, a respiration rate, an inspiration amplitude, an expiration amplitude, an inspiration-expiration ratio, a number of events per hour, a pattern of events, a sleep state, a sleep stage, pressure settings of the RPT device 122, or any combination thereof. In such a context, a sonar sensor may be understood to concern an active acoustic sensing, such as by generating and/or transmitting ultrasound and/or low frequency ultrasound sensing signals (e.g., in a frequency range of about 17-23 kHz, 18-22 kHz, or 17-18 kHz, for example), through the air.

In some implementations, the sensors 130 include (i) a first microphone that is the same as, or similar to, the microphone 140, and is integrated in the acoustic sensor 141 and (ii) a second microphone that is the same as, or similar to, the microphone 140, but is separate and distinct from the first microphone that is integrated in the acoustic sensor 141.

The RF transmitter 148 generates and/or emits radio waves having a predetermined frequency and/or a predetermined amplitude (e.g., within a high frequency band, within a low frequency band, long wave signals, short wave signals, etc.). The RF receiver 146 detects the reflections of the radio waves emitted from the RF transmitter 148, and this data can be analyzed by the control system 110 to determine a location of the user 210 (FIG. 2) and/or one or more of the sleep-related parameters described herein. An RF receiver (either the RF receiver 146 and the RF transmitter 148 or another RF pair) can also be used for wireless communication between the control system 110, the RPT device 122, the one or more sensors 130, the user device 170, or any combination thereof. While the RF receiver 146 and RF transmitter 148 are shown as being separate and distinct elements in FIG. 1, in some implementations, the RF receiver 146 and RF transmitter 148 are combined as a part of an RF sensor 147 (e.g., a RADAR sensor). In some such implementations, the RF sensor 147 includes a control circuit. The specific format of the RF communication can be Wi-Fi, Bluetooth, or the like.

In some implementations, the RF sensor 147 is a part of a mesh system. One example of a mesh system is a Wi-Fi mesh system, which can include mesh nodes, mesh router(s), and mesh gateway(s), each of which can be mobile/movable or fixed. In such implementations, the Wi-Fi mesh system includes a Wi-Fi router and/or a Wi-Fi controller and one or more satellites (e.g., access points), each of which include an RF sensor that is the same as, or similar to, the RF sensor 147. The Wi-Fi router and satellites continuously communicate with one another using Wi-Fi signals. The Wi-Fi mesh system can be used to generate motion data based on changes in the Wi-Fi signals (e.g., differences in received signal strength) between the router and the satellite(s) due to an object or person moving partially obstructing the signals. The motion data can be indicative of motion, breathing, heart rate, gait, falls, behavior, or any combination thereof.

The camera 150 outputs image data reproducible as one or more images (e.g., still images, video images, thermal images, or any combination thereof) that can be stored in the memory device 114. The image data from the camera 150 can be used by the control system 110 to determine one or more of the sleep-related parameters described herein, such as, for example, one or more events (e.g., periodic limb movement or restless leg syndrome), a respiration signal, a respiration rate, an inspiration amplitude, an expiration amplitude, an inspiration-expiration ratio, a number of events per hour, a pattern of events, a sleep state, a sleep stage, or any combination thereof. Further, the image data from the camera 150 can be used to, for example, identify a location of the user, to determine chest movement of the user 210 (FIG. 2), to determine air flow of the mouth and/or nose of the user 210, to determine a time when the user 210 enters the bed 230, and to determine a time when the user 210 exits the bed 230. In some implementations, the camera 150 includes a wide angle lens or a fish eye lens.

The infrared (IR) sensor 152 outputs infrared image data reproducible as one or more infrared images (e.g., still images, video images, or both) that can be stored in the memory device 114. The infrared data from the IR sensor 152 can be used to determine one or more sleep-related parameters during a sleep session, including a temperature of the user 210 and/or movement of the user 210. The IR sensor 152 can also be used in conjunction with the camera 150 when measuring the presence, location, and/or movement of the user 210. The IR sensor 152 can detect infrared light having a wavelength between about 700 nm and about 1 cm, for example, while the camera 150 can detect visible light having a wavelength between about 380 nm and about 740 nm.

The PPG sensor 154 outputs physiological data associated with the user 210 (FIG. 2) that can be used to determine one or more sleep-related parameters, such as, for example, a heart rate, a heart rate variability, a cardiac cycle, respiration rate, an inspiration amplitude, an expiration amplitude, an inspiration-expiration ratio, estimated blood pressure parameter(s), or a combination thereof. The PPG sensor 154 can be worn by the user 210, embedded in clothing and/or fabric that is worn by the user 210, embedded in and/or coupled to the user interface 124 and/or its associated headgear (e.g., straps, etc.), etc.

The ECG sensor 156 outputs physiological data associated with electrical activity of the heart of the user 210. In some implementations, the ECG sensor 156 includes one or more electrodes that are positioned on or around a portion of the user 210 during the sleep session. The physiological data from the ECG sensor 156 can be used, for example, to determine one or more of the sleep-related parameters described herein.

The EEG sensor 158 outputs physiological data associated with electrical activity of the brain of the user 210. In some implementations, the EEG sensor 158 includes one or more electrodes that are positioned on or around the scalp of the user 210 during the sleep session. The physiological data from the EEG sensor 158 can be used, for example, to determine a sleep state and/or a sleep stage of the user 210 at any given time during the sleep session. In some implementations, the EEG sensor 158 can be integrated in the user interface 124 and/or the associated headgear (e.g., straps, etc.).

The capacitive sensor 160, the force sensor 162, and the strain gauge sensor 164 output data that can be stored in the memory device 114 and used by the control system 110 to determine one or more of the sleep-related parameters described herein. The EMG sensor 166 outputs physiological data associated with electrical activity produced by one or more muscles. The oxygen sensor 168 outputs oxygen data indicative of an oxygen concentration of gas (e.g., in the conduit 126 or at the user interface 124). The oxygen sensor 168 can be, for example, an ultrasonic oxygen sensor, an electrical oxygen sensor, a chemical oxygen sensor, an optical oxygen sensor, a pulse oximeter (e.g., SpCh sensor), or any combination thereof. In some implementations, the one or more sensors 130 also include a galvanic skin response (GSR) sensor, a blood flow sensor, a respiration sensor, a pulse sensor, a sphygmomanometer sensor, an oximetry sensor, or any combination thereof.

The analyte sensor 174 can be used to detect the presence of an analyte in the exhaled breath of the user 210. The data output by the analyte sensor 174 can be stored in the memory device 114 and used by the control system 110 to determine the identity and concentration of any analytes in the breath of the user 210. In some implementations, the analyte sensor 174 is positioned near a mouth of the user 210 to detect analytes in breath exhaled from the mouth of the user 210. For example, when the user interface 124 is a facial mask that covers the nose and mouth of the user 210, the analyte sensor 174 can be positioned within the facial mask to monitor the mouth breathing of the user 210. In other implementations, such as when the user interface 124 is a nasal mask or a nasal pillow mask, the analyte sensor 174 can be positioned near the nose of the user 210 to detect analytes in breath exhaled through the user's nose. In still other implementations, the analyte sensor 174 can be positioned near the user 210's mouth when the user interface 124 is a nasal mask or a nasal pillow mask. In this implementation, the analyte sensor 174 can be used to detect whether any air is inadvertently leaking from the user 210's mouth. In some implementations, the analyte sensor 174 is a volatile organic compound (VOC) sensor that can be used to detect carbon-based chemicals or compounds. In some implementations, the analyte sensor 174 can also be used to detect whether the user 210 is breathing through their nose or mouth. For example, if the data output by an analyte sensor 174 positioned near the mouth of the user 210 or within the facial mask (in implementations where the user interface 124 is a facial mask) detects the presence of an analyte, the control system 110 can use this data as an indication that the user 210 is breathing through their mouth.

The moisture sensor 176 outputs data that can be stored in the memory device 114 and used by the control system 110. The moisture sensor 176 can be used to detect moisture in various areas surrounding the user (e.g., inside the conduit 126 or the user interface 124, near the user 210's face, near the connection between the conduit 126 and the user interface 124, near the connection between the conduit 126 and the RPT device 122, etc.). Thus, in some implementations, the moisture sensor 176 can be coupled to or integrated in the user interface 124 or in the conduit 126 to monitor the humidity of the pressurized air from the RPT device 122. In other implementations, the moisture sensor 176 is placed near any area where moisture levels need to be monitored. The moisture sensor 176 can also be used to monitor the humidity of the ambient environment surrounding the user 210, for example the air inside the bedroom of the user 210.

One or more Light Detection and Ranging (LiDAR) sensor 178 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having the LiDAR sensor 178 can measure and map an area extending 5 meters or more away from the sensor. The LiDAR data can be fused with point cloud data estimated by an electromagnetic RADAR sensor, for example. The LiDAR sensor(s) 178 can also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

In some implementations, the one or more sensors 130 also include a galvanic skin response (GSR) sensor, a blood flow sensor, a respiration sensor, a pulse sensor, a sphygmomanometer sensor, an oximetry sensor, a sonar sensor, a RADAR sensor, a blood glucose sensor, a color sensor, a pH sensor, an air quality sensor, a tilt sensor, a rain sensor, a soil moisture sensor, a water flow sensor, an alcohol sensor, or any combination thereof.

While shown separately in FIG. 1, any combination of the one or more sensors 130 can be integrated in and/or coupled to any one or more of the components of the system 100, including the RPT device 122, the user interface 124, the conduit 126, the humidification tank 129, the control system 110, the user device 170, the activity tracker 190, or any combination thereof. For example, the microphone 140 and the speaker 142 can be integrated in and/or coupled to the user device 170, and the pressure sensor 130 and/or the flow rate sensor 132 are integrated in and/or coupled to the RPT device 122. In some implementations, at least one of the one or more sensors 130 is not coupled to the RPT device 122, the control system 110, or the user device 170, and is positioned generally adjacent to the user 210 during the sleep session (e.g., positioned on or in contact with a portion of the user 210, worn by the user 210, coupled to or positioned on the nightstand, coupled to the mattress, coupled to the ceiling, etc.).

The data from the one or more sensors 130 can be analyzed to determine one or more sleep-related parameters, which can include a respiration signal, a respiration rate, a respiration pattern, an inspiration amplitude, an expiration amplitude, an inspiration-expiration ratio, an occurrence of one or more events, a number of events per hour, a pattern of events, a sleep state, an apnea-hypopnea index (AHI), or any combination thereof. The one or more events can include snoring, apneas, central apneas, obstructive apneas, mixed apneas, hypopneas, a mask leak, a cough, a restless leg, a sleeping disorder, choking, an increased heart rate, labored breathing, an asthma attack, an epileptic episode, a seizure, increased blood pressure, or any combination thereof. Many of these sleep-related parameters are physiological parameters, although some of the sleep-related parameters can be considered to be non-physiological parameters. Other types of physiological and non-physiological parameters can also be determined, either from the data from the one or more sensors 130, or from other types of data.

The user device 170 (FIG. 1) includes a display device 172. The user device 170 can be, for example, a mobile device such as a smart phone, a tablet, a gaming console, a smart watch, a laptop, or the like. Alternatively, the user device 170 can be an external sensing system, a television (e.g., a smart television) or another smart home device (e.g., a smart speaker(s) such as Google Home, Amazon Echo, Alexa etc.). In some implementations, the user device is a wearable device (e.g., a smart watch). The display device 172 is generally used to display image(s) including still images, video images, or both. In some implementations, the display device 172 acts as a human-machine interface (HMI) that includes a graphic user interface (GUI) configured to display the image(s) and an input interface. The display device 172 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 170. In some implementations, one or more user devices can be used by and/or included in the system 100.

The blood pressure device 182 is generally used to aid in generating cardiovascular data for determining one or more blood pressure measurements associated with the user 210. The blood pressure device 182 can include at least one of the one or more sensors 130 to measure, for example, a systolic blood pressure component and/or a diastolic blood pressure component.

In some implementations, the blood pressure device 182 is a sphygmomanometer including an inflatable cuff that can be worn by a user and a pressure sensor (e.g., the pressure sensor 132 described herein). For example, as shown in the example of FIG. 2, the blood pressure device 182 can be worn on an upper arm of the user 210. In such implementations where the blood pressure device 182 is a sphygmomanometer, the blood pressure device 182 also includes a pump (e.g., a manually operated bulb) for inflating the cuff. In some implementations, the blood pressure device 182 is coupled to the respiratory device 122 of the respiratory therapy system 120, which in turn delivers pressurized air to inflate the cuff. More generally, the blood pressure device 182 can be communicatively coupled with, and/or physically integrated in (e.g., within a housing), the control system 110, the memory 114, the respiratory therapy system 120, the user device 170, and/or the activity tracker 190.

The activity tracker 190 is generally used to aid in generating physiological data for determining an activity measurement associated with the user 210. The activity tracker 190 can include one or more of the sensors 130 described herein, such as, for example, the motion sensor 138 (e.g., one or more accelerometers and/or gyroscopes), the PPG sensor 154, and/or the ECG sensor 156. The physiological data from the activity tracker 190 can be used to determine, for example, a number of steps, a distance traveled, a number of steps climbed, a duration of physical activity, a type of physical activity, an intensity of physical activity, time spent standing, a respiration rate, an average respiration rate, a resting respiration rate, a maximum respiration rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof. In some implementations, the activity tracker 190 is coupled (e.g., electronically or physically) to the user device 170.

In some implementations, the activity tracker 190 is a wearable device that can be worn by the user 210, such as a smartwatch, a wristband, a ring, or a patch. For example, referring to FIG. 2, the activity tracker 190 is worn on a wrist of the user 210. The activity tracker 190 can also be coupled to or integrated a garment or clothing that is worn by the user 210. Alternatively still, the activity tracker 190 can also be coupled to or integrated in (e.g., within the same housing) the user device 170. More generally, the activity tracker 190 can be communicatively coupled with, or physically integrated in (e.g., within a housing), the control system 110, the memory device 114, the respiratory therapy system 120, the user device 170, and/or the blood pressure device 182.

While the control system 110 and the memory device 114 are described and shown in FIG. 1 as being a separate and distinct component of the system 100, in some implementations, the control system 110 and/or the memory device 114 are integrated in the user device 170 and/or the RPT device 122. Alternatively, in some implementations, the control system 110 or a portion thereof (e.g., the processor 112) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While system 100 is shown as including all of the components described above, more or fewer components can be included in a system according to implementations of the present disclosure. For example, a first alternative system includes the control system 110, the memory device 114, and at least one of the one or more sensors 130 and does not include the respiratory therapy system 120. As another example, a second alternative system includes the control system 110, the memory device 114, at least one of the one or more sensors 130, and the user device 170. As yet another example, a third alternative system includes the control system 110, the memory device 114, the respiratory therapy system 120, at least one of the one or more sensors 130, and optionally the user device 170. Thus, various systems can be formed using any portion or portions of the components shown and described herein and/or in combination with one or more other components.

Referring generally to FIG. 2, a portion of the system 100 (FIG. 1), according to some implementations, is illustrated. The user 210 of the respiratory therapy system 120 and a bed partner 220 are located in a bed 230 and are laying on a mattress 232. The user interface 124 (also referred to herein as a mask, e.g., a full face mask) can be worn by the user 210 during a sleep session. The user interface 124 is fluidly coupled and/or connected to the respiratory device 122 via the conduit 126. In turn, the RPT device 122 delivers pressurized air to the user 210 via the conduit 126 and the user interface 124 to increase the air pressure in the throat of the user 210 to aid in preventing the airway from closing and/or narrowing during sleep. The RPT device 122 can be positioned on a nightstand 240 that is directly adjacent to the bed 230 as shown in FIG. 2, or more generally, on any surface or structure that is generally adjacent to the bed 230 and/or the user 210.

In some implementations, the control system 110, the memory 114, any of the one or more sensors 130, or a combination thereof can be located on and/or in any surface and/or structure that is generally adjacent to the bed 230 and/or the user 210. For example, in some implementations, at least one of the one or more sensors 130 can be located at a first position 255A on and/or in one or more components of the respiratory therapy system 120 adjacent to the bed 230 and/or the user 210. The one or more sensors 130 can be coupled to the respiratory therapy system 120, the user interface 124, the conduit 126, the display device 128, the humidification tank 129, or a combination thereof.

Alternatively or additionally, at least one of the one or more sensors 130 can be located at a second position 255B on and/or in the bed 230 (e.g., the one or more sensors 130 are coupled to and/or integrated in the bed 230). Further, alternatively or additionally, at least one of the one or more sensors 130 can be located at a third position 255C on and/or in the mattress 232 that is adjacent to the bed 230 and/or the user 210 (e.g., the one or more sensors 130 are coupled to and/or integrated in the mattress 232). Alternatively or additionally, at least one of the one or more sensors 130 can be located at a fourth position 255D on and/or in a pillow that is generally adjacent to the bed 230 and/or the user 210.

Alternatively or additionally, at least one of the one or more sensors 130 can be located at a fifth position 255E on and/or in the nightstand 240 that is generally adjacent to the bed 230 and/or the user 210. Alternatively or additionally, at least one of the one or more sensors 130 can be located at a sixth position 255F such that the at least one of the one or more sensors 130 are coupled to and/or positioned on the user 215 (e.g., the one or more sensors 130 are embedded in or coupled to fabric, clothing 212, and/or a smart device worn by the user 210). More generally, at least one of the one or more sensors 130 can be positioned at any suitable location relative to the user 210 such that the one or more sensors 130 can generate sensor data associated with the user 210.

Generally, a user who is prescribed usage of the respiratory therapy system 120 will tend to experience higher quality sleep and less fatigue during the day after using the respiratory therapy system 120 during the sleep compared to not using the respiratory therapy system 120 (especially when the user suffers from sleep apnea or other sleep related disorders). For example, the user 210 may suffer from obstructive sleep apnea and rely on the user interface 124 (e.g., a full face mask) to deliver pressurized air from the respiratory device 122 via conduit 126. The respiratory device 122 can be a continuous positive airway pressure (CPAP) machine used to increase air pressure in the throat of the user 210 to prevent the airway from closing and/or narrowing during sleep. For someone with sleep apnea, their airway can narrow or collapse during sleep, reducing oxygen intake, and forcing them to wake up and/or otherwise disrupt their sleep. The CPAP machine prevents the airway from narrowing or collapsing, thus minimizing the occurrences where they wake up or is otherwise disturbed due to reduction in oxygen intake.

However, the range of positive pressures prescribed for a CPAP treatment may feel uncomfortable to a user while they are awake. In particular, in one aspect, a patient can define their comfort in two manners of input, comprising active and passive input to the system. An active indication of discomfort occurs when the patient directly provides a response to a survey and/or graphic user interface that they are experiencing breathing discomfort. Passive input as an indicator of discomfort can comprise a single measurement and/or aggregate of the measurements taken by the system (e.g. flow rate data; pressure data; waveform data; tidal volume data; minute ventilation data; and leak data), wherein a machine learning algorithm can identify measurements indicating the patient may be experiencing discomfort, resulting in adjustment by the system to alleviate discomfort. In a further aspect, the system can receive both the active and passive inputs, wherein the system will make adjustments resulting from the machine learning algorithm analysis of the active and passive inputs.

To prevent a user who is awake from experiencing these uncomfortable pressures, one solution is to provide a ramp feature that gradually increases pressure from a start pressure while the user is awake before resorting to a higher positive treatment pressure when the onset of sleep is detected. In a further aspect, if the user is awake, they can provide direct active input to the system indicating that they are in discomfort such that the ramp feature will begin to adjust. In another aspect, the system can also respond to passive input take from the associated sensor to implement the ramp feature in situations when the user is not awake or does not provide a response to an input survey.

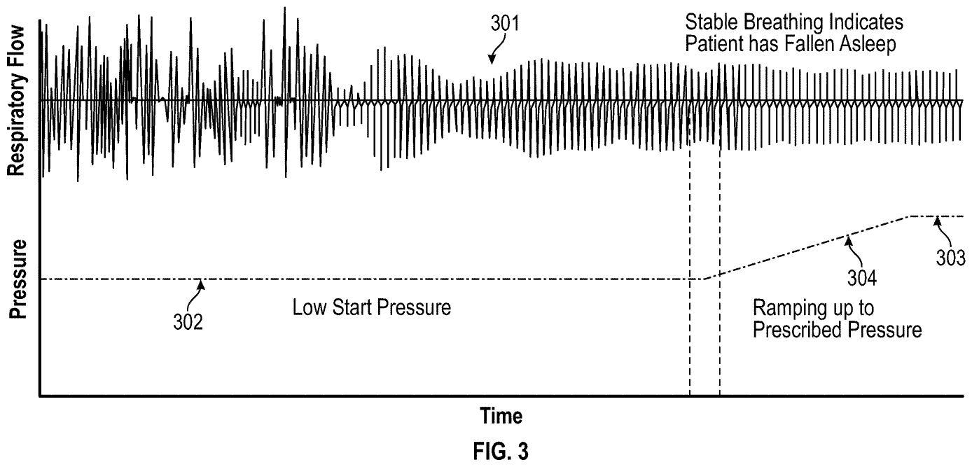

FIG. 3 includes plots showing an implementation of the ramp feature according to some aspects of the disclosure. The plots show a respiratory flow 301 over time, and a pressure over time including a start pressure 302, a prescribed pressure 303, and a pressure ramp 304 between the start pressure 302 and the prescribed pressure 303. The ramp 304 may be initiated at, or approximately at, the detection of the onset of sleep, which may be detected by the change in respiratory flow to more stable breathing. The ramp feature provides a gradual increase from the start pressure that is most comfortable to a wakeful user 210 to the pressure prescribed for their therapy. If the prescribed pressure includes a minimum and maximum pressure, the ramp 304 concludes at a prescribed minimum pressure 303.

The prescribed minimum pressure 303 may be, for example, 10 cm H2O, and is dependent on the user's therapy needs. Despite the intent of the feature, the user 210 may find the prescribed pressure 303 and/or ramp 304 actually reduces their sleep comfort rather than improving it. For example, the user 210 may find the ramp 304 increases the pressure too rapidly, making it difficult to fall asleep and/or jolting the user 210 awake. The user 210 may adjust the ramp time to provide a gentler transition from the start pressure 302 to the prescribed pressure 303.

Furthermore, some users 210 may find the ramp feature uncomfortable because of an experience of too much or too little air during the wakeful period where a start pressure 302 is provided. If there is a feeling of too little air, one way to ameliorate this feeling is to increase the start pressure 302 of the ramp, such that a higher pressure is experienced once the patient has put on the user interface 124 while awake. Alternatively, the ramp feature may be turned off completely, such that as soon as the RPT device 122 is turned on, the patient experiences the prescribed minimum pressure, which is typically higher than a prescribed start pressure. Frequently, clinicians respond to patients' complaints of a feeling of too little air by increasing the prescribed minimum pressure without altering the start pressure. However, in certain instances, increasing the prescribed minimum pressure creates a mismatch between the intended pressure increase and the actual effect.

For instance, a patient may have a therapy with a prescribed minimum pressure of 10 cm H₂O, a prescribed maximum pressure of 15 cm H₂O, and a ramp with a start pressure of 4 cm H₂O. In this example, the user will experience a gradual increase from 4 cm H₂O to 10 cm H₂O during the ramp. If this patient were to indicate that they feel like they have trouble breathing, the resultant discomfort originates from the start pressure of 4 cm H₂O during the beginning of the ramp being too low. When a clinician adjusts the minimum pressure, this fails to address the condition causing the patient to have trouble breathing, namely a start pressure that is too low. The reason for this mismatch may be clinician confusion about the difference between start pressure and minimum pressure, but in any case, it may produce a problem of patients continuing to feel like they are having trouble breathing

To help users achieve comfort early in their treatment, in one aspect, a survey is provided after a short amount of time, preferably after around three days of treatment (although other time periods may be used), to help identify any problems or difficulties the user may be experiencing and identify solutions to those problems. The data gathered from the users that participate in the survey may then be used to identify other current or future users that may experience similar difficulties.

The survey may take the form of a prompt that is transmitted to the user 210 via a patient program 430 on the user device 170 according to one or more of the following implementations: in an email (when the patient program 430 is an email client); in an SMS message; in a “notification” in the patient program 430 (when the patient program 430 is a “patient app”); in a page of a web site (when the patient program 430 is a web browser). In other implementations, the prompt may be transmitted to the display 128 of the RPT device 122 in the respiratory therapy system 120.

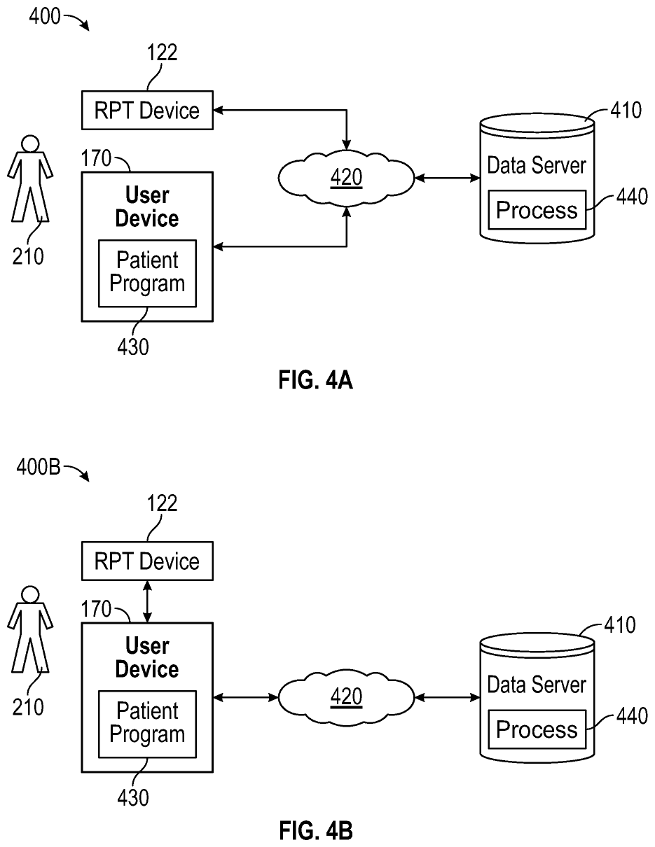

FIG. 4A contains a block diagram illustrating one implementation 400 of an RPT system according to the present technology and incorporating a data server 410, a wide area network 420, and a patient program 430 to incorporate user 210 feedback into the RPT device 122

The implementation of RPT system 400 in FIG. 4A comprises an RPT device 122 configured to provide respiratory pressure therapy to a patient 210, a data server 410, and a user device 170 associated with the patient 210. The user device 170 is co-located with the patient 210 and the RPT device 122. In the implementation 400 shown in FIG. 4A, the RPT device 122, the user device 170, and the data server 410 are connected to a wide area network 420 such as an internet or the Internet. The connections to the wide area network 420 may be wired or wireless. The user device 170 may be a personal computer, mobile phone, tablet computer, or other device. The user device 170 is configured to intermediate between the patient 210 and the data server 410 over the wide area network 420. In one implementation, this intermediation is performed by a software application program 430 that runs on the user device 170. The patient program 430 may be a dedicated application or “mobile app” that interacts with a complementary process 440 hosted by the data server 410. The process 440 may be a machine-learning model training and/or implementation, as described in more detail below. In another implementation, the patient program 430 is a web browser that interacts via a secure portal with a web site hosted by the data server 410. In yet another implementation, the patient program 430 is an email client.

FIG. 4B contains a block diagram illustrating an alternative implementation 400B of an RPT system according to the present technology. In the alternative implementation 400B, the RPT device 122 communicates with the user device 170 via a local (wired or wireless) communications protocol such as a local network protocol (e.g., Bluetooth). In the alternative implementation 400B, the local network may be identified with a local external communication network, and the user device 170 may be identified with a local external device. In the alternative implementation 400B, the user device 170, via the patient program 430, is configured to intermediate between the patient 210 and the data server 410, over the wide area network 420, and also between the RPT device 122 and the data server 410 over the wide area network 420.

In what follows, statements about the RPT system 400 may be understood to apply equally to the alternative implementation 400B, except where explicitly stated otherwise.

The RPT system 400 may contain other RPT devices (not shown) associated with respective patients who also have respective associated computing devices. All the patients in the RPT system 400 are managed by the data server 410.

The RPT device 122 is configured to store in the memory 114 therapy data from each RPT session delivered to the patient 210. Therapy data for an RPT session comprises the settings of the RPT device 122 and therapy variable data representing one or more variables of the respiratory pressure therapy throughout the RPT session.

The RPT device settings data may include:
Type of mask
Base treatment pressure (P₀)
Maximum and minimum treatment pressure limits (Pmax and Pmin)
Amplitude (A)
Humidity of delivered flow of air
Temperature of delivered flow of air

The therapy variables may include:
Respiratory flow rate (Qr)
Mask pressure (Pm)
Leak flow rate (Ql)
Tidal volume (Vt)
Measure of ventilation (Vent)
Breathing/Respiratory rate

The RPT device 122 is configured to transmit the therapy data to the data server 410. The data server 410 may receive the therapy data from the RPT device 122 according to a “pull” model whereby the RPT device 122 transmits the therapy data in response to a query from the data server 410. Alternatively, the data server 410 may receive the therapy data according to a “push” model whereby the RPT device 122 transmits the therapy data to the data server 410 as soon as convenient after an RPT session.

Therapy data received from the RPT device 122 is stored and indexed by the data server 410 so as to be uniquely associated with the RPT device 122 and therefore distinguishable from therapy data from any other RPT device(s) participating in the RPT system 400. The data server 410 also includes information regarding the user 210's clinician, such that data can be transmitted directly to the clinician. This may include a link to a user device for the clinician, or an e-mail or program link to the clinician.

The data server 410 is configured to calculate usage data for each RPT session from the therapy data received from the RPT device 122. Usage data variables for a session comprise summary statistics derived by conventional scoring means from the therapy variable data that forms part of the therapy data. Usage data may comprise one or more of the following usage variables:
Usage time, i.e. total duration of the RPT session
Apnea-hypopnea index (AHI) for the session
Average leak flow rate for the session
Average mask pressure for the session
Number of “sub-sessions” within the RPT session, i.e. number of intervals of RPT therapy between “mask-on” and “mask-off” events
Number of “mask-on” and “mask-off” events
Other statistical summaries of the therapy variables, e.g. 95th percentile pressure, median pressure, histogram of pressure values
Usage variables may comprise multi-session statistics, such as mean, median, and variance of AHI since the start of RPT therapy.

In an alternative implementation, the RPT device 122 calculates the usage variables from the therapy data stored by the RPT device 122 at the end of each session. The RPT device 122 then transmits the usage variables to the data server 410 according to the “push” or “pull” model described above.

In a further implementation, the memory 114 in which the RPT device 122 stores the therapy/usage data for each RPT session is in removable form, such as an SD memory card. The removable memory 114 may be removed from the RPT device 122 and inserted into a card reader in communication with the data server 410. The therapy/usage data is then copied from the removable memory 114 to the memory of the data server 410.

In still a further implementation, suitable for the alternative implementation 400B of the RPT system, the RPT device 122 is configured to transmit the therapy/usage data to the user device 170 via a wireless communications protocol such as Bluetooth as described above. The user device 170 then transmits the therapy/usage data to the data server 410. The data server 410 may receive the therapy/usage data from the user device 170 according to a “pull” model whereby the user device 170 transmits the therapy/usage data in response to a query from the data server 410. Alternatively, the data server 410 may receive the therapy/usage data according to a “push” model whereby the user device 170 transmits the therapy/usage data to the data server 410 as soon as it is available after an RPT session.

In some implementations, the data server 410 may carry out some post-processing of the usage data, such as with one or more processors in communication with or included in the data server 410.

The data server 410 may also be configured to receive data from the user device 170. Such may include data entered by the patient 210 to the patient program 430, or therapy/usage data in the alternative implementation 400B described above.

The data server 410 is also configured to transmit electronic messages to the user device 170. The messages may be in the form of emails, SMS messages, automated voice messages, or notifications within the patient program 430.

The RPT device 122 may be configured such that its therapy mode, or settings for a particular therapy mode, may be altered on receipt of a corresponding command via its wide area or local area network connection. In such an implementation, the data server 410 may also be configured to send such commands directly to the RPT device 122 (in the implementation 400) or indirectly to the RPT device 122, relayed via the user device 170 (in the implementation 400B).

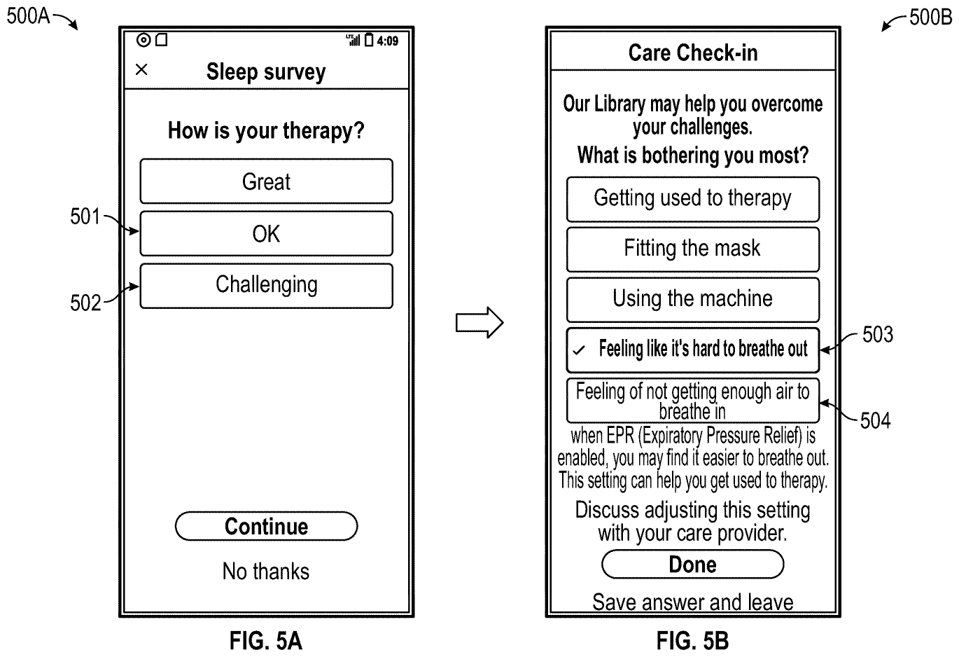

FIG. 5A depicts a graphical depiction 500A of a survey prompt being transmitted to a patient program 430 on a patient's user device 170. In an exemplary embodiment, the initial prompt is transmitted to a mobile application downloaded by the user 210 on their user device 170, preferably between two to three days after the user 210 has begun their therapy (although other time periods may be used), and asks the user 210 to answer the initial question “How is your therapy?” The user is provided three options with which to respond: “Great,” “OK,” and “Challenging.” If the user 210 selects “OK” (button 501) or “Challenging,” (button 502) the survey prompt proceeds to screen 500B shown in FIG. 5B, and provides the user 210 with a list of possible reasons that they are not completely happy with the therapy, under a heading of “What is bothering you most?” or the like. These reasons may include but are not limited to: “Feeling like it's hard to breathe out” (button 503) and “Feeling of not getting enough air to breathe in” (button 504).

If the user 210 indicates that they are having trouble breathing out by selecting button 503, the pressure applied via the face mask 124 may be too high for comfort before the user 210 has fallen asleep. This may be alleviated by directing the user 210 to enable expiratory pressure relief (EPR) or to contact a clinician to help them enable EPR. If a user selects button 503, a possible result would be to display a message explaining that enabling EPR may help as shown in FIG. 5B, such as a message reciting “When EPR is enabled, you may find it easier to breathe out. This setting can help you get used to therapy. Discuss adjusting this setting with your care provider.”

Alternatively or additionally, this may be alleviated by turning ramp 304 on if it is off, as the start pressure 302 will be lower than the pressure applied during therapy. The system 100 may be configured to perform steps to provide immediate relief in response to a user 210 selecting button 503. For example, the system 100 may be configured to automatically turn on the ramp feature in the RPT device 122 in response to a signal from user device 170 that the user 210 has selected button 503 in a patient program 430 of the user device 170. Alternatively, selecting “Feeling like it's hard to breathe out” may direct the user to instructions on how to turn on ramp 304 on their RPT device 122.

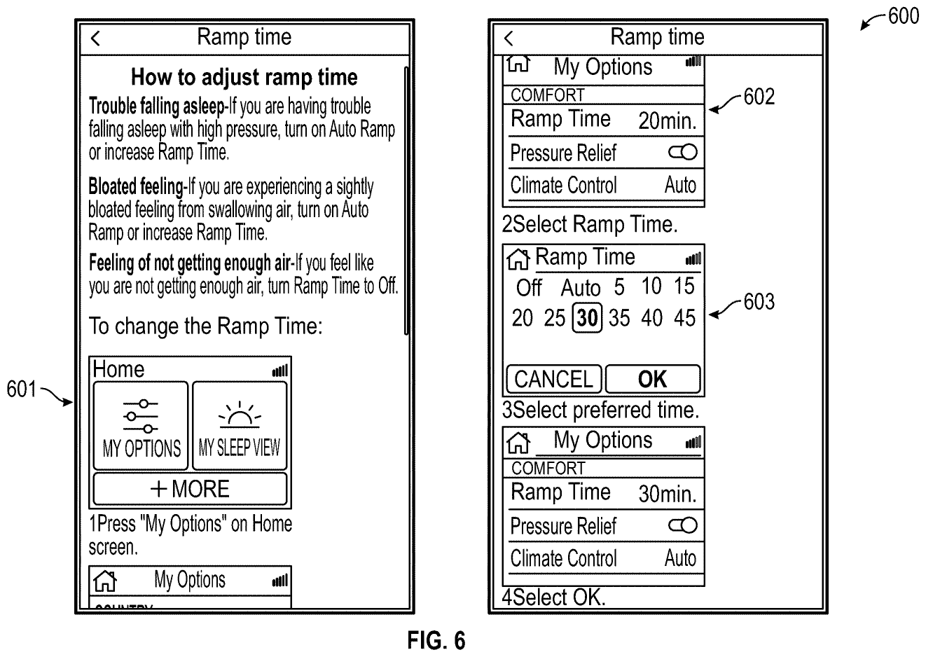

FIG. 6 is an exemplary graphical representation 600 of the instructions on how to adjust ramp on the RPT device, with guidance on what to change in accordance with the identified breathing challenge indicated by the user 210. These instructions may be displayed on the user device 170 via the patient program 430, or may be e-mailed to the user device 170. In the exemplary representation, the instructions direct the user 210 to select “my options” from the home screen 601 of the display of the RPT device 122, which directs them to an exemplary screen 602 with a set of customizable options including “ramp time.” The user is directed to tap “ramp time” on the “my options” menu, which directs them to a ramp time screen 603 that allows the user to select either “off,” “Auto,” or a specific amount of ramp time in minutes. The “Auto” feature relies on sleep detection and may trigger the ramp to minimum pressure when the user 210 has fallen asleep, as shown in the exemplary ramp feature shown in FIG. 3. If the user 210 is experiencing trouble breathing out, the user will be instructed to ensure ramp is on and/or to turn ramp on by selecting “Auto” or a specified ramp time, such that a start pressure 302 is applied during the wakeful time rather than a minimum pressure 303.

As discussed above, the user 210 may also or alternatively be directed to turn up EPR (Expiratory Pressure Relief) if they indicate that they are having trouble breathing out, or the system may do so automatically. EPR is a feature on some RPT devices that mildly lowers the pressure delivered to user 210 when exhaling. This makes it easier to breathe out against the air pressure. Selecting “Feeling like it's hard to breathe out” may direct the user 210 to exemplary directions on how to turn EPR on and/or up on their RPT device, or direct the user 210 to contact their clinician to ask for increase to EPR. The system 100 may also be configured to automatically increase EPR in the RPT device 122 in response to a signal from user device 170 that the user 210 has selected button 503 in a patient program 430 of the user device 170.

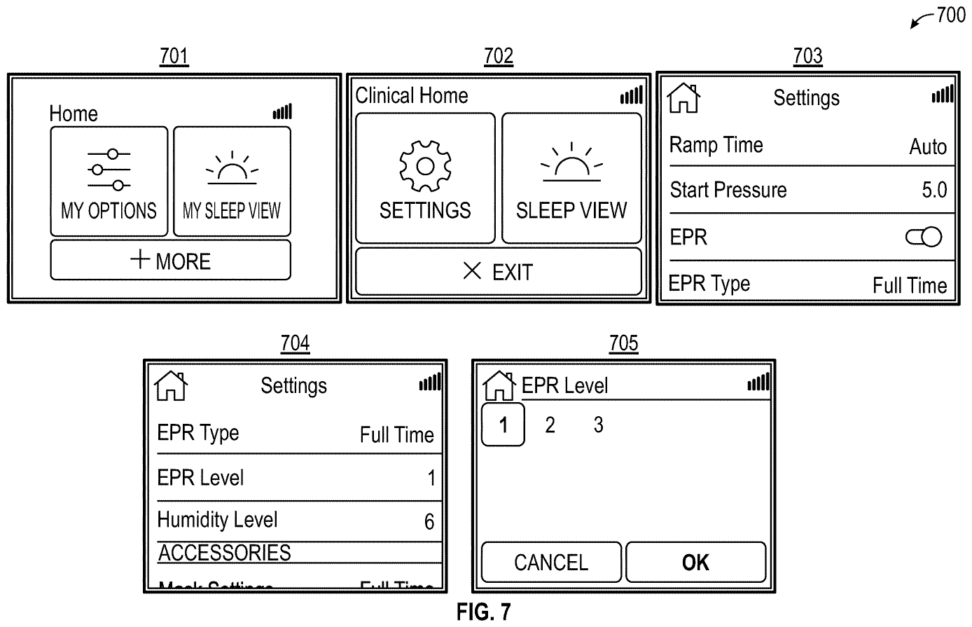

FIG. 7 is an exemplary graphical representation 700 of the instructions on how to turn EPR on when it is off, with guidance on what to change in accordance with the identified breathing challenge indicated by the user 210. The directions require advancing from the Home screen 701 and entering a “Clinical Home screen” 702 to access more advanced settings typically reserved for clinicians. In the exemplary embodiment, the Clinical Home screen 702 is accessed by a two finger tap and hold for 3 seconds, but may be accessed by other means such as a two finger swipe, etc. At the Clinical Home screen 702, tapping settings will provide a more advanced settings menu 703 that includes, among other items, the option to turn EPR on or off. The user 210 may toggle EPR on and off, and select an EPR level. Screen 704 shows further options in the settings menu 703 if the user 210 were to swipe down, including EPR level. In the exemplary embodiment, EPR level is described as levels 1, 2, or, 3 as shown in screen 705. Alternatively, the EPR level may be described as “low,” “medium,” and “high,” or in specific units of pressure, such as 1 cm H20, etc.

The user 210 may indicate that they feel like they want more air to breathe in, by pressing button 504 in FIG. 5B. This may be a result of a difference between the start pressure 302 prescribed to the user 210 and a comfortable pressure during the auto ramp feature, where the start pressure 302 experienced by the user 210 during the wakeful period before the ramp up to the prescribed pressure is too low for comfort. If the user 210 selects “I feel like I want more air to breathe in,” at button 504, the user 210 may be directed to instructions on how to improve the pressure during this time, or the system 100 may be configured to automatically adjust settings in response to the user selecting button 504.

One way to increase the pressure experienced during the waking period before falling asleep is to turn off the ramp feature. As discussed above, FIG. 6 is an exemplary graphical representation 600 of the instructions on how to adjust ramp on the RPT device. These instructions may be displayed on the user device 170 via the patient program 430, or may be e-mailed to the user device 170 in response to the user selecting “I feel like I'm having trouble breathing in” (button 504) at the exemplary screen 500B. In the exemplary representation, the instructions direct the user 210 to select “my options” from the home screen 601 of the display of the RPT device 122, which directs them to an exemplary screen 602 with a set of customizable options including “ramp time.” The user is directed to tap “ramp time” on the “my options” menu, which directs them to a ramp time screen 603 that allows the user to select either “off,” “Auto,” or a specific amount of ramp time in minutes. If the user 210 is experiencing a feeling that they want more air, the user will be instructed to turn the ramp off by selecting “Off.” This has the effect of applying the prescribed minimum pressure 303 during the wakeful period before sleep onset rather than the typically lower start pressure 302. The system 100 may also be configured to turn ramp off automatically in response to a user 210 selecting button 504, via communication between the user device 170, RPT device 122, and/or network 420. A signal indicating that the user 210 has selected button 504 on the user device 170, optionally via a patient program 430, may be transmitted to RPT device 122 to turn ramp off without further user 210 input.

Alternatively to, or in conjunction with, the above instructions to turn the ramp off, the user may be given instructions to select a preferred start pressure for the ramp feature. This feature is accessed through the more advanced settings menu described in the exemplary embodiment of FIG. 7. The directions require advancing from the Home screen 701 entering a “Clinical Home screen” 702 to access more advanced settings typically reserved for clinicians. In the exemplary embodiment, the clinical Home screen 702 is accessed by a two finger tap and hold for 3 seconds, but may be accessed by other means such as a two finger swipe, etc. At the Clinical Home screen 702, tapping settings will provide a more advanced settings menu 703 that includes, among other items, the option to adjust a start pressure 302. The start pressure may be adjusted using terms like “low,” “medium, and “high” or using numerical values from lowest to highest, such as “1” for low and “5” for high and the values 2 through 4 representing intermediate pressures, or using precise pressure values in known units, such as cm H₂O. The user 210 may be directed to increase the start pressure on the ramp feature using these tools. In an alternative aspect, an external monitor, such as a clinician or server, may be alerted that patient is experiencing air hunger. Air hunger can comprise a patient feeling discomfort due to the sensation of not receiving enough air. In response, a start pressure associated with the system needs to be increased, requiring a settings change to be implemented. Further, the clinician can specify predetermine thresholds for measurement settings such that the system can adjust and implement automatic settings changes (e.g. switching ramp off, increase minimum pressure) that may be pushed to the patient if air hunger is detected.

In a further aspect, an assessment of the patient's breathing status can be categorized into at least three classifications comprising: 1) the user experiencing steady-state breathing discomfort, 2) the user not receiving sufficient air during breathing, and 3) the user not expelling sufficient air during breathing. The threshold defining sufficient air will be relative to the patient based on certain biomarkers comprising at least: age, height, weight and/or BMI, gender, level of physical fitness, and overall health. The biomarkers along with measurements such a flow rate data; pressure data; waveform data; tidal volume data; minute ventilation data; and leak data, can be provided to a model. The results from the model can determine whether sufficient air is being received or expelled such that the system can make adjustments to the operation of the system to alleviate the patient feeling discomfort for insufficient air during inhalation and exhalation.

In addition to discomfort experience during inhalation and exhalation, the patient may experience discomfort not related directly to inhalation and exhalation but instead to a steady-state feeling of discomfort. During steady-state discomfort, the patient may experience that they have a constant feeling of air hunger such that they are not receiving enough air. The steady-state discomfort experienced by the patient can be similarly assessed as the discomfort related to inhalation and exhalation. The identifying of the difference in experiences can be done by the patient providing active direct responses to the surveys. For example, the survey questions can ask under which instances does the patient feel they are not receiving enough air (e.g. during inhale, during exhale and/or a constant feeling of not having enough air). Similar to the approach to remedying discomfort from insufficient air during inhalation and exhalation, the input received from the surveys associated with steady-state discomfort can be paired with the passive sensor measurements and provided to a model for responsive therapy by the system.

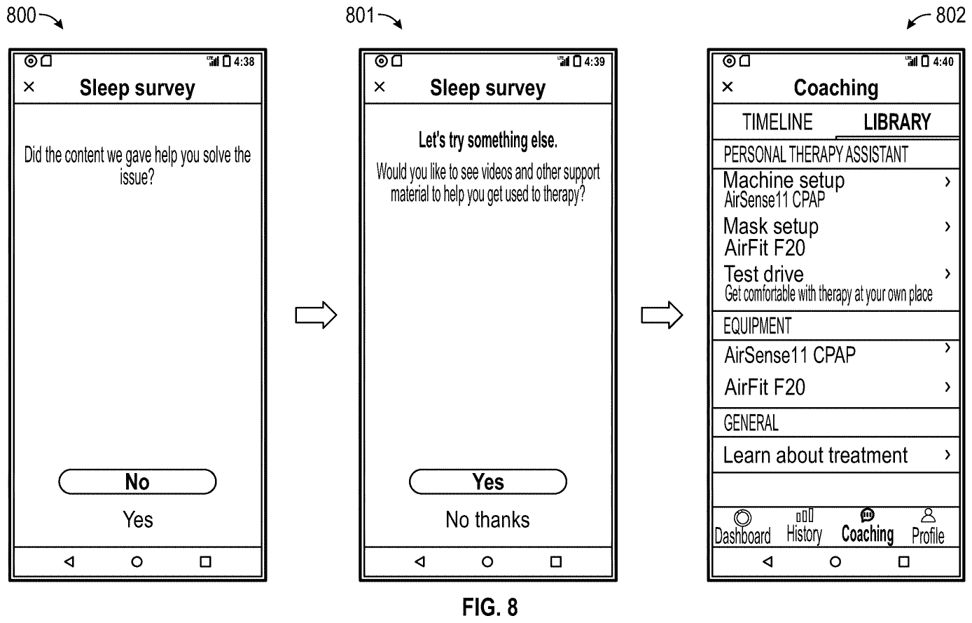

After a user 210 has engaged with the survey questions described in FIGS. 5A and 5B, a follow up self-help prompt may be provided to determine if the information provided to the user 210 was helpful in overcoming the problem they identified. As shown in FIG. 8, the prompt may take the form of a simple yes or no question such as “Did the content we gave help you solve the issue?” transmitted to an interface 800 of the user device 170 or RPT device 122. The question may be transmitted through the patient program 430. The user's answer to this prompt may be paired with data gathered from the user's system 100 to identify the problem the user had experienced and the resolution they attempted to overcome the problem. Furthermore, the prompt and answer will provide data as to whether the attempted resolution did or did not solve the problem identified by the user 210. As shown in screens 801 and 802, other possible solutions may further be offered. If the user 210 engages in any other potential solutions, this activity may be tracked, and the follow-up self-help screen 800 may be applied again after that solution has been attempted to further provide data as to activities that help alleviate problems identified by users 210

Advantageously, high resolution data may be collected from patients using the RPT devices 122. This data may include breath waveforms with flow rate (respiratory flow) data, tidal volume data, pressure data, minute ventilation data, leak data, inspiratory to expiratory (IE) ratio, and other signals from sensors and transducers that may provide some indication of breathing discomfort and the correlation with the pressure settings thereof. The sensors 130 described in FIG. 1 may be used to gather this data. Measures of ventilation may include one or both of inspiratory and expiratory flow, per unit time. When expressed as a volume per minute, this quantity is often referred to as “minute ventilation.” Minute ventilation is sometimes given simply as a volume, understood to be the volume per minute.

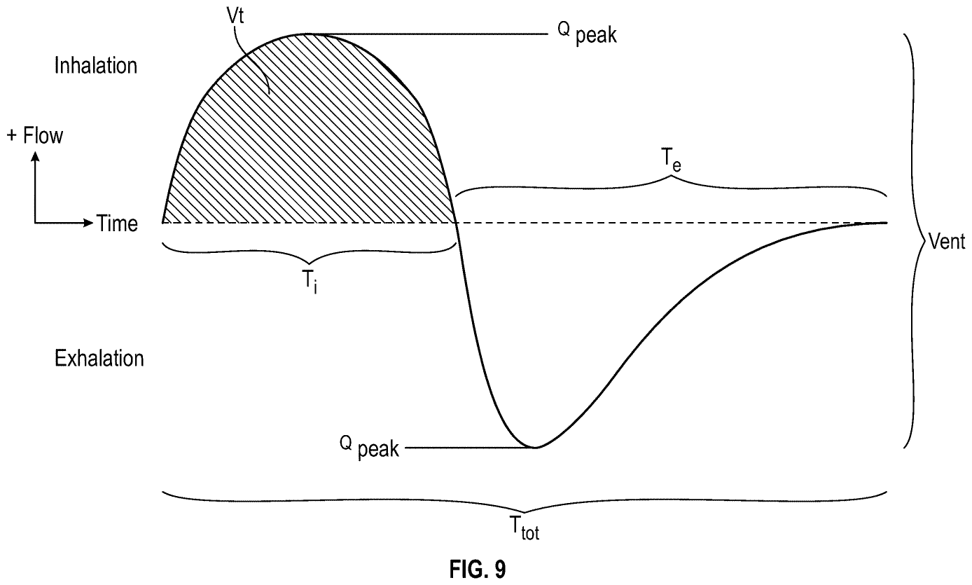

FIG. 9 shows a model typical breath waveform of a person and at least some of the data that may be available in determining patients that may have trouble breathing and/or uncomfortable breathing. The horizontal axis is time, and the vertical axis is respiratory flow rate. While the parameter values may vary, a typical breath may have the following approximate values: tidal volume Vt of 0.5 L, inhalation time Ti 1.6 s, peak inspiratory flow rate Qpeak 0.4 L/s, exhalation time Te 2.4 s, peak expiratory flow rate Qpeak −0.5 L/s. The total duration of the breath, Ttot, is about 4 s. The person typically breathes at a rate of about 15 breaths per minute (BPM), with Ventilation Vent about 7.5 L/min. A typical duty cycle, the ratio of Ti to Ttot, is about 40%.

This data may be coupled with the user responses to the survey and with other data gathered by the sensors described in FIG. 1 and with other user data (e.g. demographic information (age, gender, weight, height, BMI, etc.), patient reported outcome measures (PROMS) data such as sleepiness improvements or motivations to start therapy, the type of sleep test that the user used to identify or diagnose their apnea (e.g., a polysomnography (PSG) or home sleep test (HST)) to create training data for a machine learning model for identifying users 210 that may experience breathing problems, including users that experience breathing discomfort, for example a feeling that it is hard to breathe out or a desire for more air flow. The model may be used to identify other patients that may be having similar problems but have not engaged with the survey. For example, the machine learning model may pre-emptively identify patients having similar problems even before the first survey during their therapy. In some embodiments, the machine learning model may be able to identify patients who may experience discomfort breathing in or out before they even start therapy.

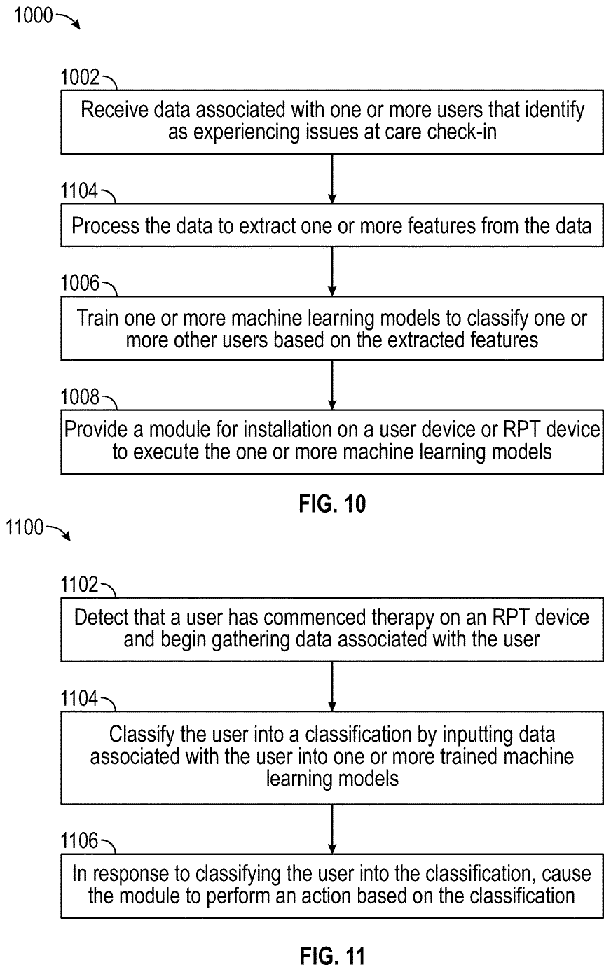

FIG. 10 depicts a flowchart of an exemplary process 1000 for training a machine-learning model to learn associations between data obtained from one or more users 210, according to certain embodiments.

As used herein, a “machine-learning model” generally encompasses instructions, data, and/or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine-learning model is generally trained using training data, e.g., experiential data and/or samples of input data, which are fed into the model in order to establish, tune, or modify one or more aspects of the model, e.g., the weights, biases, criteria for forming classifications or clusters, or the like. Aspects of a machine-learning model may operate on an input linearly, in parallel, via a network (e.g., a neural network), or via any suitable configuration.

The execution of the machine-learning model may include deployment of one or more machine learning techniques, such as linear regression, logistical regression, random forest, gradient boosted machine (GBM), deep learning, and/or a deep neural network. Supervised and/or unsupervised training may be employed. For example, supervised learning may include providing training data and labels corresponding to the training data, e.g., as ground truth. Unsupervised approaches may include clustering, classification or the like. K-means clustering or K-Nearest Neighbors may also be used, which may be supervised or unsupervised. Combinations of K-Nearest Neighbors and an unsupervised cluster technique may also be used. Any suitable type of training may be used, e.g., stochastic, gradient boosted, random seeded, recursive, epoch or batch-based, etc.

It should be understood that techniques according to this disclosure may be adapted to any suitable type of activity. It should also be understood that the examples above are illustrative only. The techniques and technologies of this disclosure may be adapted to any suitable activity.

Presented below are various aspects of machine learning techniques that may be adapted to identify whether users 210 may be experiencing discomfort in their therapy. As will be discussed in more detail below, machine learning techniques adapted to identify users 210 that may be experiencing discomfort may include one or more aspects according to this disclosure, e.g., a particular selection of training data, a particular training process for the machine-learning model, operation of a particular device suitable for use with the trained machine-learning model, operation of the machine-learning model in conjunction with particular data, modification of such particular data by the machine-learning model, etc., and/or other aspects that may be apparent to one of ordinary skill in the art based on this disclosure.

As discussed in further detail below, the one or more components of an exemplary environment or system 100 may generate, store, train and/or use a machine-learning model. The exemplary environment or system 100 or one of its components may include a machine-learning model and/or instructions associated with the machine-learning model, e.g., instructions for generating a machine-learning model, training the machine-learning model, using the machine-learning model, etc. The exemplary environment or system 100 or one of its components may include instructions for retrieving data, adjusting data, e.g., based on the output of the machine-learning model, and/or operating a display to output data, e.g., as adjusted based on the machine-learning model. The exemplary environment or system 100 or one of its components may include, provide, obtain, and/or generate training data.

In some embodiments, a system or device other than the components shown in the exemplary system 100 may be used to generate and/or train the machine-learning model. For example, such a system may include instructions for generating and/or obtaining the machine-learning model, the training data and ground truth, and/or instructions for training the machine-learning model. A resulting trained-machine-learning model may then be provided to the exemplary system 100 or one of its components and, for example, stored in the memory 114.

Generally, a machine-learning model includes a set of variables, e.g., nodes, neurons, filters, etc., that are tuned, e.g., weighted or biased, to different values via the application of training data. In supervised learning, e.g., where a ground truth is known for the training data provided, training may proceed by feeding a sample of training data into a model with variables set at initialized values, e.g., at random, based on Gaussian noise, a pre-trained model, or the like. The output may be compared with the ground truth to determine an error, which may then be back-propagated through the model to adjust the values of the variable. Certain embodiments may utilize, for training a machine learning model, unsupervised learning where, e.g., the sample of training data may not include pre-assigned labels or scores to aid the learning process or may utilize semi-supervised learning where a combination of training data with pre-assigned labels or scores and training data without pre-assigned labels or scores is used to train a machine learning model.

FIG. 10 depicts a flowchart of an exemplary process 1000 for training a machine-learning model to learn to identify users 210 experiencing discomfort during therapy, according to certain embodiments. At step 1002, the process 1000 may include receiving data associated with one or more users 210. For example, data server 410 may receive data from system 100, including data from control system 110, respiratory therapy system 120, sensor(s) 130, user device 170, blood pressure device 182, and/or activity tracker 190. The data associate with the users 210 may also include demographic data (such as age, gender, height, weight), PROMS data, type of sleep test and other data a machine may learn to identify as factors.

Data from user device 170 includes a user's 210 responses to the survey questions shown in FIGS. 5-8. The user's answers to these questions help to produce a self-identifying batch of users 210 that are experiencing discomfort or other issues during their therapy.

At step 1004, the process 1000 includes processing the data to extract one or more features from the data. This may include creating batches of data for input into the machine-learning model. Batches may include but are not limited to:
(1) Users who identified therapy as “OK”
(2) Users who identified therapy as “Challenging”
(3) Users who identified “Feeling like it's hard to breathe out” as bothering them most
(3a) Users who responded by increasing EPR
(3b) Users who responded by turning ramp on
(4) Users who identified “Feeling of not getting enough air to breathe in” as bothering them most.
(4a) Users who responded by turning ramp off
(4b) Users who responded by decreasing EPR
(4c) Users who responded by adjusting (e.g., increasing/decreasing) the start pressure on the ramp feature, the minimum pressure, EPR, etc.
(5a) Users whose therapy improved with the adjustment
(5b) Users whose therapy did not improve despite the adjustment
(6) Users who identified therapy as “Great”

The data of some users may be included in more than one batch and the batches are not mutually exclusive. For example, a user that identified as feeling “OK” will have their data included in batch (1). If they indicated that they had trouble breathing out, their data would also be included in batch (3). Also, the machine-learning model may mix and combine batches to create new training data. As just one example, it may identify users who identified therapy as “Challenging,” identified “Feeling of not getting enough air to breathe in” as bothering them most, responded by turning ramp off, and reported that the adjustment improved their therapy into a new batch for processing data.

The data from system 100 is then associated with the users in each batch. Advantageously, this may associate specific empirical and sensor-based data with users 210 that self-identify as having specific issues or none at all. The data may include flow rate (respiratory flow) data, tidal volume data, pressure data, minute ventilation data, leak data, the breathing waveform and data associated with the breathing waveform as described above, and/or other data and signals accessed by sensors 130 and which may provide some relevance or potential relevance to breathing discomfort. Additional data may include the pressure settings of the RPT device 122.

At step 1006, this collected and processed data is used to train one or more machine learning models to classify one or more other uses based on the data from their systems 100, including sensors 130, blood pressure device 182, and/or activity tracker 190. This may be accomplished by associating sensor data with the self-identified users as described in step 1004, and may produce multiple models for different batches as identified above. Training may be conducted in any suitable manner, e.g., in batches, and may include any suitable training methodology, e.g., stochastic or non-stochastic gradient descent, gradient boosting, random forest, etc. In some embodiments, a portion of the training data may be withheld during training and/or used to validate the trained machine-learning model, e.g., may be used to compare the output of the trained model with the ground truth for that portion of the training data to evaluate an accuracy of the trained model. The training of the machine-learning model may be configured to cause the machine-learning model to learn associations between training data (e.g., user data) and ground truth data, such that the trained machine-learning model is configured to determine an output (e.g., the user is experiencing a feeling like it's hard to breathe) in response to the input data (e.g., sensor data associated with that user), based on the learned associations.

In various embodiments, the variables of a machine-learning model may be interrelated in any suitable arrangement in order to generate the output. For example, in some embodiments, the machine-learning model may include an architecture that is configured to classify users based on the user's breathing data and/or RPT data gathered from sensors described in FIGS. 1-2, the user responses to the survey prompts described in FIGS. 5-8, and/or other data and the like. For example, the machine-learning model may include one or more neural networks configured to identify features in the data, and may include further architecture, e.g., a connected layer, neural network, etc., configured to determine a relationship between the identified features in order to determine patterns in the data. In certain embodiments, the machine learning model may include a single node for classification, as described elsewhere herein. The trained model, for example, may determine that certain breathing waveforms, combined with particular values of tidal volume data and leak data, results in a user experiencing a feeling that it's hard to breathe, and identifies that the user may benefit from an adjustment to the ramp start pressure or other modifications to comfort settings, such as a change in humidification level or EPR, or a combination of these and other settings. The trained model may determine that certain breathing waveforms, combined with particular values of tidal volume data and leak data, results in a user experiencing difficulty breathing out, and identifies that the user may benefit from an adjustment to the EPR or other modifications to comfort settings, such as a change in humidification level, temperature, or ramp parameters, or a combination of these and other settings. These are just two examples of many classifications that may be determined by the machine-learning model. Other comfort settings may include changes to elements of the air circuit, such as mask type, mask fit, tube length, or tube diameter.

At step 1008, the trained models are incorporated into a module or other processor means for installation on one or more of the user device 170, the RPT device 122, any other suitable location within system 100 or RPT system 400/400B. The module or other processor may be used to execute the one or more machine learning models. The module may take the form of a sub-program within the patient program 430 described in FIGS. 4A and 4B that may take the form of a mobile application, it may be a stand-alone program outside of the patient program 430, or it may be a program module incorporated into the RPT device 122. As shown in FIGS. 4A and 4B, the one or more machine-learning models may be stored as a process 440 on a separate data server 410, and the module for installation may just be an executable program for communicating with the process 440 via a wide-area network 420.

The module may be provided a web server device for publication of the module for download and installation using a user device, or may provide the software module to the user device 170 or the RPT device 122 directly for installation, and/or the like. The software module may include one or more program files, electronic applications, and/or the like that can be installed on the user device 170 and executed by the user device 170. For example, the software module may include data for installation of a virtual assistant and the one or more machine learning models on a web browser of the user device 170 or within a patient program 430 on the user device 170.

Accordingly, certain embodiments may train one or more machine learning models to classify one or more users based on their data alone or in combination with answers to the survey described in FIGS. 5-8. The process 1000 described above is provided merely as an example, and may include additional, fewer, different, or differently arranged steps than depicted in FIG. 10.

FIG. 11 illustrates an exemplary process 1100 for identifying users that may be experiencing discomfort or other issues during therapy, e.g., by utilizing a trained machine-learning model such as a machine-learning model trained according to one or more embodiments discussed above. At step 1102, the process 1100 may include detecting that a user 210 has initiated therapy on an RPT device 122. This may be accomplished by a program within system 100, optionally within RPT device 122, which indicates when it is in use, by an indication from user 210 via patient program 430, by a wireless signal sent from RPT device 122 to a remote location, including a location having the machine-learning model, or by any other suitable means. When the user 210 begins therapy, system 100 begins gathering data about the patient's therapy via sensors 130, blood pressure device 182, and/or activity tracker 190.

At step 1104, the data gathered about the therapy is input into the one or more machine-learning models trained in process 1000. The data may be input into the model continuously in real-time, periodically at predetermined time intervals, on-demand, or in any other time interval. The data may be transmitted from system 100 or user device 170 to data server 410 to be processed in the machine learning model at process 440. When the user data gathered at step 1102 is input into the trained machine-learning model, the model may classify the user based on the batches used in the training. For example, the trained model may identify if the user 210 would likely have identified the therapy as “Great,” “OK,” or “Challenging” based on the model's decision (as dictated by the training data). Furthermore, the model may classify the user 210 as likely to experience trouble breathing in, likely to have trouble breathing out, and may classify the user 210 as likely to benefit from a specific therapeutic intervention, such as an instruction to turn ramp on or turn ramp off, etc. As discussed above, the trained model, for example, may determine that certain breathing waveforms, alone or in combination with particular values of tidal volume data, leak data and/or other signals that may provide an indication of breathing discomfort, results in a user experiencing a feeling that it's hard to breathe, and identifies that the user may benefit from an adjustment to the ramp start pressure, minimum pressure or other changes to comfort settings. Alternatively, the trained model may determine that certain breathing waveforms, combined with particular values of tidal volume data leak data and/or other signals that may provide an indication of breathing discomfort, results in a user experiencing difficulty breathing out, and identifies that the user may benefit from an adjustment to the EPR or other changes to comfort settings. These are just two examples of many classifications that may be determined by the machine-learning model and identifications of actions to execute in response. The other signals that may provide an indication of breathing discomfort may include mask on/off events, that may indicate that a patient 210 has removed a mask during therapy. This may be an indication that the patient 210 was experiencing discomfort. Alternatively, the amount of time it takes a patient to fall asleep (time from putting on the mask to sleep onset) may also be an indication of breathing comfort as it may foreseeably take a person longer to fall asleep if they are uncomfortable.

At step 1106, in response to the classification performed in step 1104, the process 1100 may cause the module to perform an action based on the classification. These actions may include but are not limited to: sending a notification to the user that a modification to their therapy may make them more comfortable, automatically making the adjustment to the RPT device 122 (including changes to ramp, pressure settings, EPR, etc.) that the model identifies as beneficial based on the user's classification, sending a notification to the clinician of user 210 informing the clinician of a potential identified problem in the user's therapy, etc.

Based on the gathered data and information, the model may indicate that a particular mask change, whether in mask size or mask type, would be beneficial to the patient 210. If recommending a mask change, the recommendation may be either sent directly to the user 210, or to the user's clinician directing the clinician that the user 210 may benefit from a mask change. Similarly, if recommending a change in tube type, the recommendation may be either sent directly to the user 210, or to the user's clinician directing the clinician that the user 210 may benefit from a tube change.

For example, if the model determines that a user 210 is experiencing breathing waveforms combined with tidal volume figures and leak numbers or other indications that may result in the user 210 experiencing a feeling of not getting enough air to breathe in, the process 1100 may cause the module to transmit a signal to RPT device 122 to turn the ramp feature off. This may be done independently of the user 210 engaging in the survey described in FIGS. 5-8. In this way, the machine-learning model allows for users 210 that do not or have not engaged in the survey to benefit from the data gathered from users 210 who have.

The survey prompt described in FIG. 5A may be sent to all users 210 of the RPT device 122 regardless of the classification determined by the machine-learning model. However, not all users 210 will engage with the survey process. One or more aspects of process 1100 may be performed whether the user 210 has performed the survey process or not. The engaged users 210 may thereby provide additional training data so that all users may benefit from the survey prompts.

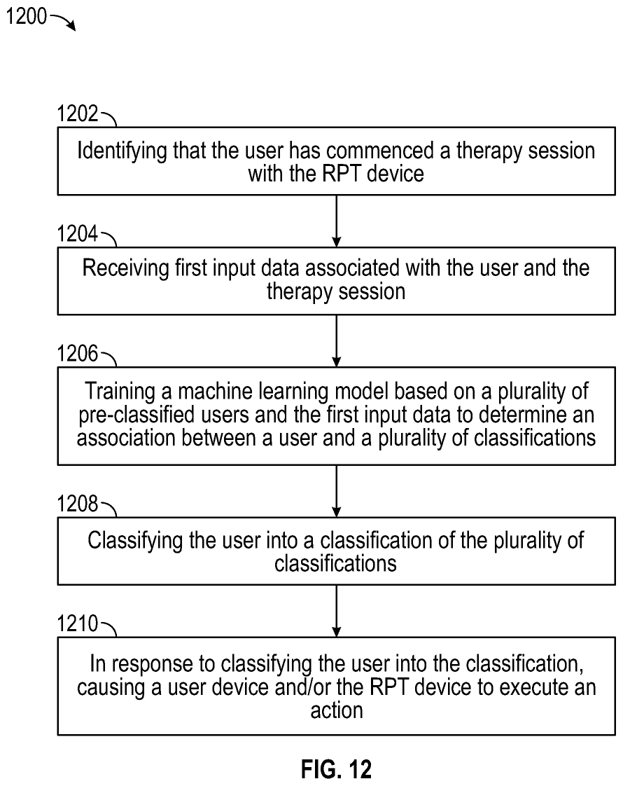

FIG. 12 illustrates an alternative exemplary process 1200 for identifying users that may be experiencing discomfort or other issues during therapy, e.g., by utilizing a trained machine-learning model such as a machine-learning model trained according to one or more embodiments discussed above. At step 1202, the process 1200 may include detecting that a user 210 has initiated therapy on an RPT device 122. This may be accomplished by a program within system 100, optionally within RPT device 122, which indicates when it is in use, by an indication from user 210 via patient program 430. At step 1204, the process may include receiving first input data associated with the user and the therapy session. At step 1206, the process 1200 may include training a machine learning model based on a plurality of pre-classified users and the first input data to determine an association between a user and a plurality of classifications. At step 1208, the process 1200 may include, classifying the user into a classification of the plurality of classifications. At step 1210, the process 1200 in response to classifying the user into the classification, may include causing a user device and/or the RPT device to execute an action.

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Furthermore, “approximately”, “substantially”, “about”, or any similar term used herein means+/−5-10% of the recited value.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior disclosure. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilized to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

What is claimed is:

1. A computer-implemented method for classifying a user of a respiratory pressure therapy (RPT) device, comprising:
identifying that the user has commenced a therapy session with the RPT device;
receiving first input data associated with the user and the therapy session;
training a machine learning model based on a plurality of pre-classified users and the first input data to determine an association between a user and a plurality of classifications;
classifying the user into a classification of the plurality of classifications; and
in response to classifying the user into the classification, causing a user device and/or the RPT device to execute an action.

2. The method of claim 1, further comprising:
transmitting a survey prompt to a user;
receiving one or more selections from the user in response to the survey prompt, wherein at least response is an indication of user discomfort; and
automatically adjusting one or more of settings of the RPT based on the indication of user discomfort.

3. The method of claim 1, wherein the first input data includes at least one of: flow rate data; pressure data; waveform data; tidal volume data; minute ventilation data; and leak data.

4. The method of claim 1, wherein the plurality of classifications include:
the user experiencing steady-state breathing discomfort,
the user not receiving sufficient air during breathing, and
the user not expelling sufficient air during breathing.

5. The method of claim 4, wherein the action includes displaying directions to alleviate not receiving sufficient air during breathing.

6. The method of claim 5, wherein the action includes the RPT device automatically turning a ramp feature off.

7. The method of claim 4, wherein the action includes displaying directions to alleviate the user not expelling sufficient air during breathing.

8. The method of claim 7, wherein the directions include the RPT device automatically increasing an expiratory pressure relief (EPR).

9. A non-transient computer-readable storage medium having instructions embodied thereon, the instructions being executable by one or more processors to perform a method for training a machine-learning model for classifying a user of a respiratory pressure therapy (RPT) device, the method comprising:
receiving input data associated with users and associated therapy sessions, wherein each user has one or more classifications;
processing the input data to extract one or more features from the input data;
training the machine learning model to classify one or more other users into the one or more classifications, based on the one or more feature extracted from the input data; and
providing communication between the machine learning model and one or both of a user device and the RPT device, wherein the communication comprises at least causing a user device and/or the RPT device to execute an action.

10. The non-transient computer-readable storage medium of claim 9, further comprising instructions executable to perform the method:
transmitting a survey prompt to a user;
receiving one or more selections from the user in response to the survey prompt, wherein at least response is an indication of user discomfort; and
automatically adjusting one or more of settings of the RPT based on the indication of user discomfort.

11. The non-transient computer-readable storage medium of claim 9, wherein the receiving of the input data associated with the user and the therapy session is received from the user device and/or the RPT device.

12. The non-transient computer-readable storage medium of claim 9, wherein the input data includes at least one of: flow rate data; tidal volume data; minute ventilation data; and leak data.

13. The non-transient computer-readable storage medium of claim 9, wherein the one or more classifications include:
the user experiencing steady-state breathing discomfort,
the user not receiving sufficient air during breathing, and
the user not expelling sufficient air during breathing.

14. The non-transient computer-readable storage medium of claim 13, wherein the action includes displaying directions to alleviate not receiving sufficient air during breathing.

15. The non-transient computer-readable storage medium of claim 14, wherein the one action includes automatically turning a ramp feature off.

16. The non-transient computer-readable storage medium of claim 14, wherein the action includes displaying directions on the RPT device to alleviate the user not expelling sufficient air during breathing.

17. The non-transient computer-readable storage medium of claim 16, wherein the directions include the RPT device automatically increasing an expiratory pressure relief (EPR).

18. A system for classifying a user of a respiratory pressure therapy (RPT) device, comprising:
a memory storing instructions and a machine learning model trained based on pre-classified users so as to learn associations between the pre-classified users and a plurality of classifications, such that the machine learning model is configured to perform the classifying based on the learned associations; and
a processor operatively connected to the memory and configured to execute the instructions to perform operations including:
identifying that the user has commenced a therapy session on the RPT device;
receiving first input data associated with the user and the therapy session;
classifying the user into a classification of the plurality of classifications by inputting the first input data into the machine learning model; and
in response to classifying the user into the classification, causing the user device and/or the RPT device to execute an action.

19. The system of claim 18, wherein the instructions are further configured to:
transmit a survey prompt to a user;
receive one or more selections from the user in response to the survey prompt, wherein at least response is an indication of user discomfort; and
automatically adjust one or more of settings of the RPT based on the indication of user discomfort.

20. The system of claim 18, wherein the plurality of classifications include:
the user experiencing steady-state breathing discomfort,
the user not receiving sufficient air during breathing, and
the user not expelling sufficient air during breathing.
